(19) United States
(12) Patent Application Publication
(10) Pub. No.: US 2025/0221830 A1
(43) Pub. Date: Jul. 10, 2025

(54) SYSTEMS AND METHODS FOR REDUCTION AND FIXATION OF FRACTURES AND TISSUES

(71) Applicant: Primo Medical Group, Stoughton, MA (US)

(72) Inventor: Steven J. Tallarida, Mansfield, MA (US)

(21) Appl. No.: 19/009,323

(22) Filed: Jan. 3, 2025

Related U.S. Application Data
(60) Provisional application No. 63/682,689, filed on Aug. 13, 2024.
(60) Provisional application No. 63/618,147, filed on Jan. 5, 2024.

(51) Int. Cl. A61F 2/46

(52) U.S. Cl. CPC A61F 2/46; A61F 2002/4635

(57) ABSTRACT

Capture and fixation systems and methods for fracture reduction and fixation and/or tissue capture and fixation. A first implant is coupled to an elongated, flexible tension component via an attachment component of the first implant. The first implant and the flexible tension component are inserted into and fixed to a first bone portion. The flexible tension component is adjusted so as to position the first bone portion and a second bone portion in a reduced fracture configuration. A second implant is inserted into and affixed to the second bone portion. The second implant engages the flexible tension component, which causes tension in the flexible tension component that holds the first and second bone portions in the reduced fracture configuration. The second implant can also further capture a tissue and fixate it to the second bone portion.

### CROSS-REFERENCE TO RELATED APPLICATIONS

This application is a non-provisional and claims benefit of U.S. Provisional Application No. 63/682,689 filed Aug. 13, 2024 and U.S. Provisional Application No. 63/618,147 filed Jan. 5, 2024, the specification(s) of which is/are incorporated herein in their entirety by reference.

### FIELD OF THE INVENTION

The present invention relates to systems and methods for treatment or fixation of bone fractures and/or tissues.

### BACKGROUND OF THE INVENTION

In fracture repair, it is generally appreciated that fractured segments must be first reduced to be in close proximity to one another, and then be fixed in place somewhat rigidly to allow the biologic mechanisms of bone repair to occur. In non-unions, or fractures that resist healing, it is generally appreciated that reduction, fixation, or systemic, metabolic, or vascularity problems are at fault.

In a non-limiting example, a fracture in consideration is the commonly fractured scaphoid bone of the hand, although other fractures such as the clavicle are equally suited for this approach. Referring to FIGS. 1-2, the scaphoid bone is found distal and somewhat medial to the radius of the arm. A non-union of a scaphoid fracture is reported in the medical literature as a fairly common complication. Rates of non-union are presented anywhere from 10% up to 50% in certain series. These non-unions are believed to be a function of both poor approximation of the fractured segments as well as vascularity of the bony structures after the reduction. Non-unions and vascular problems resulting from the fracture can lead to necrosis of this bone or collapse. Both of these conditions can result in dysfunction of the wrist and may lead to wrist arthritis which requires surgical procedures, such as a four-corner fusion, a proximal row carpectomy, or wrist arthroplasty.

Multiple devices have been introduced to try to improve the fixation of this fragment, but most of these devices were developed in the 1990's and early 2000's and are based on bone screw configurations that are headless so that they can be buried in the bone to prevent head protrusion of the screw, which interferes with movement or creates wear on opposed anatomy or creates a discomfort for the patient. Devices that implement external fixation can lead to complications such as site infection, osteomyelitis, malunion, failure or loosening, soft-tissue impalement, compartment syndrome, and neurovascular injury.

Fracture of the scaphoid is commonly treated today using these headless compression screws in which there is a distal threaded segment and a proximal threaded segment. These two threaded segments are at different thread pitches, so as they are driven into place, a form of compression is created between the two segments along the unthreaded shaft. Alternatively, a screw with a variable pitch over the length of its thread also attempts to create a form of compression over the length of the screw. One can appreciate the challenge of sizing and driving these screws into fragments with the hope that they are positioned correctly axially and have generated some degree of compression between the fragments at the fracture plane. This challenge is further exacerbated if the fracture plane is not more or less perpendicular to the long axis of the screw.

Surgically, these devices have additional technical challenges. These screws are generally placed below the cortical surface of the bone. Passing different pitch threads through the same region of cancellous bone, can compromise the pathway cut into the bone and ultimately compromise the screw purchase and fixation into the bone. Also, as will be clearer in the diagrams, the working axis of these screws and their delivery techniques may create insurmountable challenges in getting an optimum reduction of the fractured segments.

As shown in FIG. 2, the scaphoid bone has a bulbous, proximal and distal section and a reduced midsection or waist. Fracture of this bone is typically through the thinner waist section. However, this fracture may also be non-perpendicular to the long axis, oblique, or spiral, in which cases the reduction can be much more challenging.

FIGS. 3-4 are schematic renditions of the fractured scaphoid showing a fracture separating the bone into a distal segment (4a) and a proximal segment (4b). FIG. 5 illustrates the typical surgical repair is to drive a guide wire (8) into either the distal or proximal portion of the bone and then advance it into the remaining segment. The fracture shown demonstrates that there is an angular displacement (5) as well as an axial displacement (6) of the fracture. There may be a portion of the fragment which has not completely fractured through or maybe minimally displaced relative to the other fracture segment.

It is important to note that once the guide wire (8) has been placed through both segments, a constraint has been developed such that the two bone segments are now fixed along the axis of the guide wire (8). Further reduction of these two segments will now be limited as to the first surfaces that make physical contact as the two segments are drawn together. In traditional treatment of these fractures, the subsequent delivery of the various compression screw embodiments, will not allow for the correction of this misalignment, i.e., the gap as shown in FIG. 6 cannot be completely reduced as the fragments are making contact. Hence, there is a need for a new treatment that will allow for a more complete reduction of the bone fracture.

### BRIEF SUMMARY OF THE INVENTION

It is an objective of the present invention to provide novel systems and methods to reduce fractures in a manner that allows for a more accurate approximation of the fractured components, allows for a minimal access approach of fracture reduction, and allows for a greater working distance when trying to reduce a fractured component which may be substantially displaced from another segment, as specified in the independent claims. Embodiments of the invention are given in the dependent claims. Embodiments of the present invention can be freely combined with each other if they are not mutually exclusive.

In some embodiments, the present invention features a tissue or fracture fixation system comprising a flexible tension component, a first implant comprising an attachment component configured to couple the tension component with the first implant, and a second implant configured to engage the tension component to secure the first implant relative to the second implant. Without wishing to limit the present invention, the tension component allows for fracture reduction of a bone fragment relative to a second bone fragment.

In some embodiments, the first implant may further comprise a lumen configured for use with a guidewire. The attachment component may be offset from a central axis of the lumen. The tension component may be coupled and routed through the first implant such that the tension component does not interfere with the lumen when used with the guidewire. In some embodiments, the first implant may be radially symmetric. In other embodiments, the first implant may further comprise exterior threads. The first implant can engage an interior of a bone. For example, the first implant can be anchored or fixated to the interior of a bone. The interior of the bone may be a bone tunnel.

In a non-limiting embodiment, the first implant may comprise a first component comprising the attachment component and a rounded or blunted tip, and a second component having a threaded exterior surface, a lumen, and at least two grooves disposed along the length of the lumen. The attachment component is offset from a central axis of the lumen and aligns with the grooves. The tension component may be disposed within the grooves to prevent obstruction of the lumen.

In another embodiment, the first implant may comprise a first component comprising the attachment component, and a second component having a rounded or blunted tip, a threaded exterior surface, and the lumen. The attachment component may be offset from a central axis of the lumen to prevent obstruction of the lumen. In some embodiments, the second component may be configured to mate with the first component. As a non-limiting example, the first component and the second component can be press-fitted to each other. In other embodiments, the attachment component may be a U-shaped groove. The attachment component can act as a pulley mechanism that engages the tension component.

In some embodiments, the second implant may comprise a lumen through which the tension component is disposed, and an opening coupled to the lumen. The opening may be a tapering wedge from the proximal end into an interior of the lumen. The opening of the second implant may be sized to receive and capture the compressible locking component. In other embodiments, the second implant may comprise a V-shaped notch disposed on its distal end. The V-shaped notch may be configured to capture a tissue, a suture, or suture tape.

In other embodiments, the tension component may comprise a compressible locking component configured to be disposed within the second implant. The compressible locking component may be a sliding knot bundle. When tension is applied to the tension component, the tapering of the second implant prevents the compressible locking component from going further inside the lumen, and further causes the compressible locking component to collapse upon itself such that the compressible locking component secures the first implant relative to the second implant.

In some other embodiments, the second implant has a tapering outer diameter such that the second implant has a reduction in size at its distal end. The second implant may be configured to capture and drive a tissue, a suture, or suture tape into a hole. Without wishing to limit the present invention, the tapered distal end allows for easier insertion of said tissue, suture, or suture tape into said hole.

In some embodiments, the second implant has a threaded surface, barbs, or annular protrusions on its outer surface, and/or a ferrule disposed at its proximal end. In some embodiments, the second implant is T-shaped and comprises T-arms disposed at its proximal end. The T-shaped arms may be configured to anchor tissue. In some other embodiments, the second implant is L-shaped and comprises an arm disposed at its proximal end for anchoring tissue.

In some embodiments, the fixation system may include at least one supporting implant disposed between the first implant and the second implant. The tension component may be disposed through a lumen of the supporting implant or adjacent to the supporting implant. In some other embodiments, the first implant, the second implant, the supporting implant, or a combination thereof is treated with one or more anti-clotting, antithrombogenic coatings or surface treatments, or nanotechnology treatments to stimulate bone growth.

In some embodiments, the fixation system may further comprise a driver tool for implanting the first implant and the second implant. The driver tool may have a drive shaft having a channel or lumen for receiving the tension component and a drive distal tip. The drive distal tip may comprise a square or star head. In other embodiments, the driver tool may further comprise a handle connected to the drive shaft. The handle can have a storage compartment and grooves that lead to said storage compartment. The storage compartment may be configured to retain at least the second implant, and the grooves may be configured to retain at least a portion of the tension component prior to use. In some embodiments, the driver tool may further comprise a ferrule connected to the drive shaft. The ferrule is configured to retain the second implant prior to use.

In some embodiments, the handle comprises a receiving area disposed within the handle and intersecting a pathway for the tension component. In some embodiments, the system may further comprise a winder tool configured to be inserted into the receiving area of the handle, and capture and wind the tension component. As the tension component is wound onto the winder tool, tension is applied to the tension component.

In some embodiments, the present invention features a tissue or fracture fixation system comprising a flexible tension component, a first implant comprising an attachment component configured to couple the tension component to the first implant, and a second implant configured to engage the tension component so as to secure the first implant relative to the second implant. In other embodiments, the system comprises a first implant coupled to a flexible tension component by looping the tension component around a catch groove of the first implant. The first implant and the flexible tension component is inserted through a second bone portion and fixed to a first bone portion. The flexible tension component is adjusted so as to position the first bone portion and the second bone portion in a reduced fracture configuration in which a gap or displacement between the first bone portion and the second bone portion is reduced. A second implant is inserted into and affixed to the second bone portion. The second implant further engages the flexible tension component, which causes tension in the flexible tension component that holds the first bone portion and the second bone portion in the reduced fracture configuration.

In yet other embodiments, the tissue or fracture fixation system of the present invention may comprise a first implant, at least one elongated, flexible tension component attached to and extending from the first implant, and a second implant having a capture mechanism configured to engage the tension component, thus securing the first implant relative to the second implant.

According to some embodiments, the present invention also features a method of treating a bone fracture between a first bone portion and a second bone portion. The method may comprise implanting a first implant that is coupled to a tension component, via an attachment component, into the first bone portion, manipulating the flexible tension component so as to reduce the bone fracture between the first bone portion and the second bone portion, and implanting a second implant in the second bone portion such that the second implant engages the flexible tension component so as to hold the first bone portion and the second bone portion in place.

One of the unique and inventive technical features of the present invention is the attachment component coupling the flexible tension component to the first implant. Without wishing to limit the present invention to a particular theory or mechanism, the attachment component is a means for attaching the tension component to the first implant in a manner that allows for cannulation and provides a pivot mechanism. Furthermore, the present invention also makes reduction of fractures through limited exposures possible. The system would also provide a one size fits most approach to numerous fracture reduction scenarios, which would eliminate numerous part codes and inventory costs which have previously been required for these procedures. None of the presently known prior references or works has the unique inventive technical feature of the present invention.

Another unique and inventive technical feature of the present invention is the anchoring of the first implant to the interior of the bone, such as the bone tunnel. Without wishing to be bound to a particular theory or mechanism, this configuration allows for the first implant to be better secured to the bone and increases tensile strength. Furthermore, by fixing the first implant inside the bone, there are less complications as compared to external fixation. None of the presently known prior references or works has the unique inventive technical feature of the present invention.

According to other embodiments, the present invention features a method of anchoring a first tissue portion to a second tissue portion. The method may comprise implanting a first implant that is coupled to a tension component, via an attachment component, into the first tissue portion, and implanting at least a portion of a second implant into the second tissue portion. In other embodiments, the implanting step may comprise applying tension to the tension component such that the second implant engages a compressible locking component of the tension component, which causes the compressible locking component to collapse upon itself and secure the second implant relative to the first implant.

In some embodiments, the second implant engages and secures the tension component in place. The second implant may further capture a third tissue portion and fixate it to the second tissue portion. In other embodiments, the first tissue portion and the second tissue portion may be bone, and the third tissue portion may be a ligament or tendon.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows the bones of the wrist, including the scaphoid bone.

FIG. 2 is a close-up view of the scaphoid.

FIG. 3 is a schematic rendition of the scaphoid.

FIG. 4 is a schematic rendition of a fractured scaphoid.

FIG. 5 shows a typical surgical repair of a fractured scaphoid using a guide wire.

FIG. 6 is a side view of the fractured scaphoid and the guide wire.

FIG. 7 shows a pilot hole created within the fractured scaphoid.

FIG. 8A is an exploded view of a first implant of the present invention.

FIG. 8B is another exploded view of the first implant coupled with a suture.

FIG. 8C is an assembled view of the first implant.

FIG. 9A is a top view of the first implant showing the suture pathways.

FIG. 9B is a bottom view of the first implant with a suture.

FIGS. 10A-10D shows a reduction system of the present invention comprising the first implant, a second implant, and suture being used for fracture repair.

FIG. 11A is an alternative embodiment of the second implant having a notched distal end.

FIG. 11B shows a cross-sectional view of the second implant with the notched distal end and an interior channel that expands at the distal and proximal ends of the second implant.

FIG. 11C shows a side cross-sectional view of the second implant.

FIGS. 12A and 12B show how the notch end of the second implant can be used to capture tendons.

FIGS. 13A-13D illustrate how the notch end of the second implant can be used to capture sutures and anchor to a bone fragment or a different bone.

FIG. 14A shows another embodiment of the second implant.

FIG. 14B shows a cross-sectional view of the second implant with an interior channel that tapers from the proximal end to the distal end of the second implant.

FIG. 14C shows the second implant inserted into the same bone with the first implant.

FIG. 14D shows the second implant inserted into an adjacent bone separate from the first implant.

FIGS. 15A-15C shows the second implant of FIG. 11A being used with a driver tool that is inserted into the proximal end of the second implant.

FIGS. 16A-16C shows an embodiment of the driver tool having a handle that can store the second implant coupled to a suture.

FIG. 17A shows another embodiment of the reduction system that includes a support implant.

FIG. 17B shows the handle storing the second implant and support implant that are coupled to a suture.

FIG. 18A-18C shows a winder tool being used with the driver tool to tighten the sutures.

FIGS. 19A-19B shows an alternative embodiment of the driver tool.

FIGS. 20A-20B shows another alternative embodiment of the second implant having a T-configuration.

FIGS. 21A-21B demonstrates use of the T-shaped second implant.

FIGS. 22A-22B shows the T-shaped second implant coupled to tissue.

FIGS. 23A-23D shows another alternative embodiment of the second implant having an L-configuration.

FIG. 24A-24B shows how to insert the first implant into the bone (sutures not shown for clarity).

FIG. 24C shows the sutures coupled to the first implant inserted into the bone.

FIG. 25A-25B shows how the second implant is used to capture and fixate soft tissue to the bone.

FIG. 26A-26B shows how the second implant is used to capture and fixate soft tissue to the bone.

FIG. 27A is an exploded view of a first implant and a driver device of the present invention.

FIG. 27B is an assembled view of the first implant and the driver device.

FIG. 28 is a side view of the first implant.

FIG. 29A shows a distal component and a proximal component of the first implant with a groove feature.

FIG. 29B shows the distal component and the proximal component with a suture in the groove feature.

FIG. 30A is a bottom view of the first implant without a suture.

FIG. 30B is a bottom view of the first implant with a suture.

FIG. 31 shows the proximal component.

FIG. 32 is an assembled view of the first implant.

FIG. 33A is a side view of the first implant and delivery device.

FIG. 33B is a side view of the first implant and delivery device with a suture.

FIG. 34A is a cross-sectional view of the first implant and delivery device.

FIG. 34B is a cross-sectional view of the first implant and delivery device with a suture.

FIG. 35A is another view of the first implant and the delivery device with the suture.

FIG. 35B illustrates the delivery driver being removed from the first implant.

FIG. 36A shows the guide wire in the scaphoid fragments with the first implant positioned to be driven into the scaphoid fragments.

FIG. 36B is a section view showing the first implant driven into the distal bone fragment.

FIG. 36C shows the delivery driver being removed from the first implant that remains within the distal bone fragment.

FIG. 37A shows the distal bone fragment placed in a reduced position relative to the proximal bone fragment by pulling on the sutures.

FIG. 37B shows a second implant positioned to be driven into the proximal bone fragment.

FIG. 37C shows the reduction system fully assembled with the fracture reduced.

FIG. 38A shows an alternative embodiment with a midsection implant placed after the first implant and sutures disposed therethrough.

FIG. 38B shows the reduction system fully assembled for the embodiment with the midsection implant.

FIG. 39 shows one embodiment of the midsection implant.

FIG. 40 shows one embodiment of the second implant.

FIG. 41 shows an auxiliary implant used in conjunction with the reduction system.

FIG. 42A shows the auxiliary implant connected to an auxiliary implant driver.

FIG. 42B shows a distal tip of the auxiliary implant driver.

FIG. 42C is an alternative embodiment of the distal tip of the auxiliary implant driver.

FIG. 43A shows the auxiliary implant driven into a separate portion of the bone.

FIG. 43B shows a second auxiliary implant driven into another portion of the bone.

FIG. 44A is a side view of one auxiliary implant in the bone.

FIG. 44B is a side view of multiple auxiliary implants in the bone.

FIG. 44C is a top view of one auxiliary implant in the bone.

FIG. 44D is a top view of multiple auxiliary implants in the bone.

FIG. 45 shows a non-limiting embodiment of a fracture plate system that may be used in accordance with the present invention.

FIG. 46 shows an alternative embodiment of the first implant with a blunted tip.

FIG. 47 shows an alternative embodiment of the second implant for securing the sutures.

FIG. 48 shows an alternative embodiment of a first implant with a zip-tie.

FIG. 49 shows an alternative embodiment of a second implant for securing the zip-tie.

FIG. 50 shows another embodiment of the second implant for securing the zip-tie.

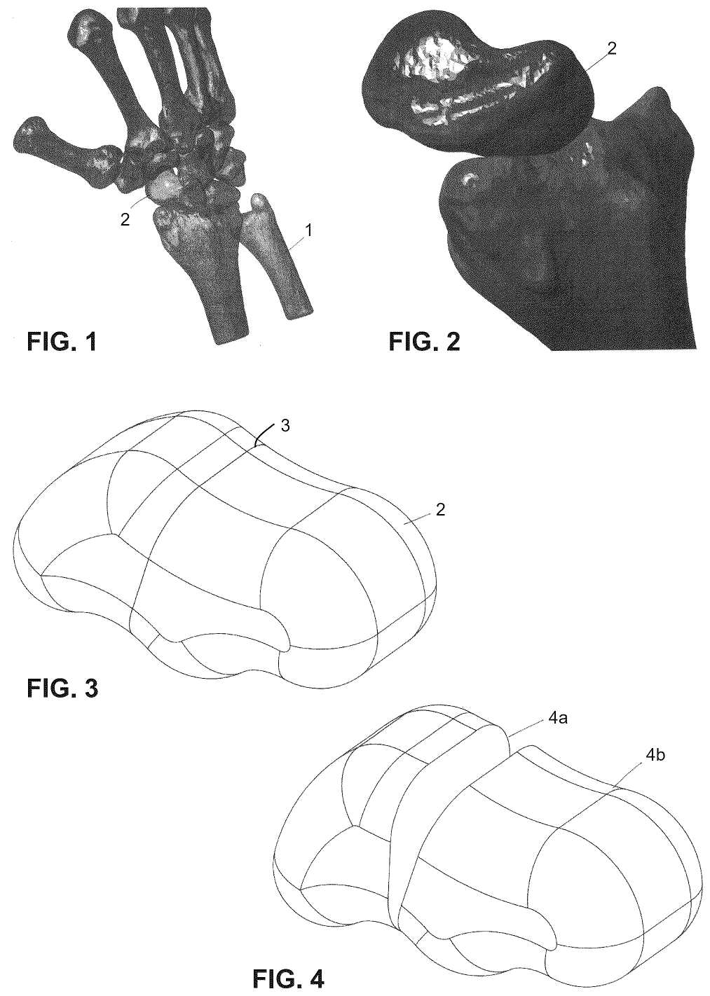

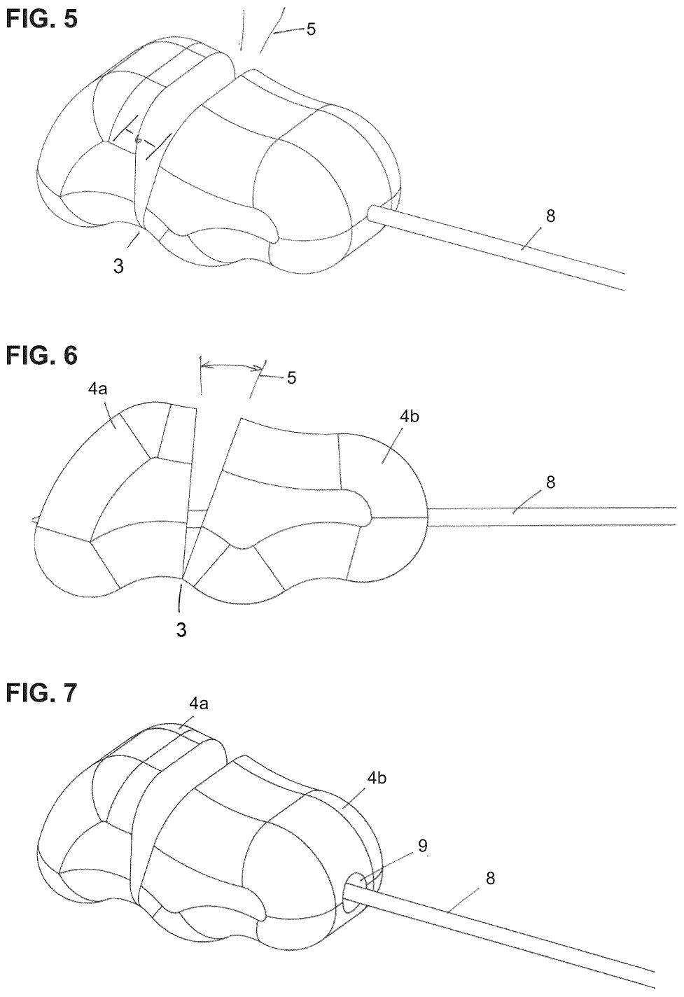

### DETAILED DESCRIPTION OF THE INVENTION

The following is a list of reference numbers corresponding to a particular element referred to herein:
2—bone
3—fracture
4a—distal portion
4b—proximal portion
5—angular displacement of fracture
6—axial displacement
7a—distal tunnel
7b—proximal tunnel
8—guide wire
9—pilot hole
24—traverse
30—closed gap
100—reduction system
10, 52, 110—first implant
11a, 111—first component, or alternatively, distal component
112—rounded tip
11b, 113—second component, or alternatively, proximal component
114—vents
15—cutting edge
16—cutting and thread tap cutting features
21, 116—lumen
18, 118—attachment component, U-shaped groove, or catch groove
17, 19, 20, 120—proximal grooves, pathway
51—zip-tie
53—zip-tie end
33, 62, 130—second implant
34, 60, 132—distal end of second implant
35, 134—proximal end of second implant
39, 136—lumen of second implant
40—auxiliary implant
41—proximal tip
42—attachment section
43—distal cutting tip
44—auxiliary implant driver
45—cutting tip
46—distal end of driver
47—reduced diameter area
48—tri-lobe interface
49—implant angle
50—implant angle
54—receiving slot
55—zip-tie lock
56—flange
57—flat surface
58—bulge surface
59—boss
61—receiving slot
63—locking element
70—fracture plate
138—ferrule, rim, or arm
140—V-shaped notch
22, 150—tension component
23a, 152a—tension limb; suture segment
23b, 152b—tension limb; suture segment
155—slipknot
160—static suture
12, 200—driver tool
13a—distal element
14—access slots
203—shaft
205—handle
13b, 210—driver tube or channel
215—handle grooves
217—storage compartment
250—winder
255—receiving area
260—driver ferrule
36, 300—supporting implant
37, 305—outer surface of supporting implant
38, 310—lumen of supporting implant
350—tendons or ligaments

Referring to FIG. 6, the guide wire (8) has been placed through the proximal portion (4b) and advanced into the distal portion (4a), which constrains the two bone portions along the axis of the guide wire (8). Further reduction of these two portions will be limited as to the first surfaces that make physical contact as the two portions are drawn together. Subsequent delivery of previous compression screw embodiments will not allow for the gap to be completely reduced as the fragments are making contact. Without wishing to be limited to a particular theory or mechanism, the system of the present invention overcomes the limitations of prior devices.

As used herein, the term “tissue” includes hard tissue such as bone; firm tissue such as cartilage; and soft tissue such as muscles, tendons, and ligaments. Tendons, ligaments, and muscles are connective tissues that can attach to bone and other organs.

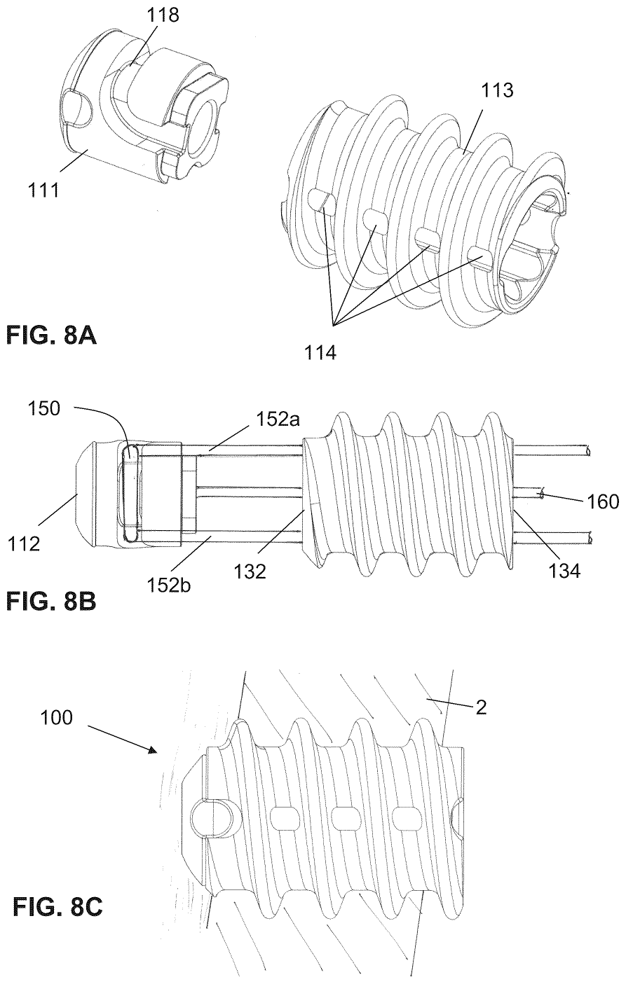

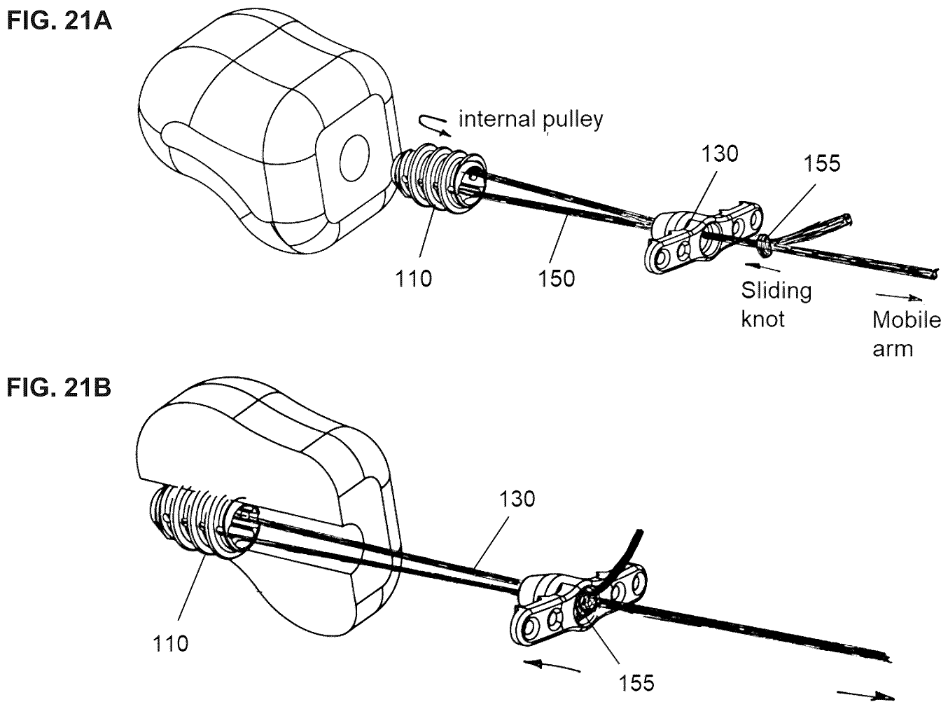

Referring now to FIGS. 8A-23D, the present inventions features a tissue or fracture fixation system (100) comprising a flexible tension component (150), a first implant (110) comprising an attachment component (118) configured to couple the tension component (150) with the first implant (110), and a second implant (130) configured to engage the tension component (150) to secure the first implant (110) relative to the second implant (130).

In some embodiments, the first implant (110) may further comprise a lumen (116) configured for use with a guidewire. The attachment component (118) may be offset from a central axis of the lumen. The tension component (150) may be coupled and routed through the first implant such that the tension component (150) does not interfere with the lumen (116) when used with the guidewire.

In some embodiments, the first implant (110) may be radially symmetric. In other embodiments, the first implant (110) may further comprise exterior threads. The first implant (110) can engage an interior of a bone. For example, the first implant (110) can be anchored or fixated to the interior of a bone. The interior of the bone may be a bone tunnel.

In a non-limiting embodiment, the first implant (110) may comprise a first component (111) comprising the attachment component (118) and a rounded or blunted tip (112), and a second component (113) having a threaded exterior surface, a lumen, and at least two grooves disposed along the length of the lumen. The attachment component (118) is offset from a central axis of the lumen and aligns with the grooves. The tension component (150) may be disposed within the grooves to prevent obstruction of the lumen (116).

In another non-limiting embodiment, the first implant (110) may comprise a first component (111) comprising the attachment component (118), and a second component (113) having a rounded or blunted tip (112), a threaded exterior surface, and the lumen (116). The attachment component (118) may be offset from a central axis of the lumen to prevent obstruction of the lumen (116).

In some embodiments, the second component (113) may be configured to mate with the first component (111). As a non-limiting example, the first component (111) and the second component (113) can be press-fitted to each other. In other embodiments, the attachment component (118) may be a U-shaped groove.

In other embodiments, the second implant (130) may comprise a lumen (136) through which the tension component (150) is disposed. The lumen (136) can be sized to minimally clear the tension component. The second implant (130) can have an opening coupled to the lumen. The opening tapers by reducing in size from an exterior to an interior of the second implant (130). In some embodiments, the opening of the second implant (130) is sized to receive and capture the compressible locking component (155). The opening can taper in diameter into an interior of the lumen. For example, the opening diameter may be about 1.5-2.5 times larger than the interior diameter of the lumen.

In other embodiments, the opening may be a tapering wedge from the proximal end into an interior of the lumen. In some embodiments, the interior diameter of the second implant (130) gradually narrows such that a taper angle at the proximal end (134), α, and a taper angle at the distal end (132), β, ranges from about 5-25°.

In some embodiments, the tension component (150) may comprise a compressible locking component (155) configured to be disposed within the second implant (130). The compressible locking component (155) may be a sliding knot bundle. When tension is applied to the tension component (150), the tapering of the second implant prevents the compressible locking component (155) from going further inside the lumen, and further causes the compressible locking component (155) to collapse upon itself such that the compressible locking component (155) secures the first implant (110) relative to the second implant (130).

In other embodiments, the second implant (130) may comprise a V-shaped notch (140) disposed on its distal end (132). Without wishing to limit the invention, the V-shaped notch (140) may be configured and/or utilized to capture a tissue, a suture, or suture tape. In further embodiments, the second implant (130) may have a tapering outer diameter such that the second implant has a reduction in size at its distal end (132). For example, the distal end (132) of the second implant may be conical or pointed. In some embodiments, the second implant is configured to capture and drive a tissue, a suture, or suture tape into a hole. Without wishing to limit the invention, the tapered distal end (132) allows for easier insertion of said tissue, suture, or suture tape into said hole.

In some embodiments, the system may further comprise a static tension component (160) coupled to the distal component (111). The static tension component (160) may be disposed along a third groove of the second component (113).

In other embodiments, the second implant (130) may have a threaded surface, barbs, or annular protrusions on its outer surface. In some other embodiments, the second implant (130) may have a ferrule (138) disposed at its proximal end (134). In some embodiments, the second component (113) comprises side vent holes (114).

In still other embodiments, the second implant (130) comprises T-arms (138) disposed at its proximal end (134) such that the second implant (130) is T-shaped. Without wishing to limit the invention, the T-shaped arms (138) are configured to anchor tissue (350) to another tissue or surface.

In alternative embodiments, the second implant (130) has an arm (138) disposed at its proximal end (134) for anchoring tissue (350) such that the second implant (130) is L-shaped. Without wishing to limit the invention, the arm (138) may be used to anchor tissue (350) to another tissue or surface.

In some embodiments, the system (100) may further comprise at least one supporting implant (300) disposed between the first implant (110) and the second implant (130). The tension component (150) may be disposed through a lumen (310) of the supporting implant or adjacent to the supporting implant (300). The supporting implant (300) may comprise surface treatments and nanotechnology treatments to stimulate bone growth. In other embodiments, the first implant (110), the second implant (130), at least one supporting implant (300), or a combination thereof is treated with one or more anti-clotting, antithrombogenic coatings.

In some embodiments, the attachment component (118) couples the tension component (150) to the first implant (110) to allow for cannulation. The attachment component (118) can act as a pulley mechanism that engages the tension component (150). Without wishing to limit the present invention to a particular theory or mechanism, when the first implant (110) is implanted in a bone fragment, the tension component (150) allows for fracture reduction of the bone fragment relative to a second bone fragment.

In some embodiments, the system may further comprise a driver tool (200) for implanting the first implant (110) and the second implant (130). The driver tool (200) may have a drive shaft (203) having a channel or lumen for receiving the tension component (150) and a drive distal tip. The drive distal tip may comprise a square or star head. In other embodiments, the driver tool (200) may further comprise a handle (205) connected to the drive shaft (203). The handle (205) can have a storage compartment (217) and grooves (215) that lead to said storage compartment (217). The storage compartment (217) may be configured to retain at least the second implant (130), and the grooves (215) may be configured to retain at least a portion of the tension component (150) prior to use. In alternative embodiments, the driver tool (200) may further comprise a ferrule (260) connected to the drive shaft (203) and configured to retain the second implant (130) prior to use.

In some embodiments, the handle (205) comprises a receiving area (255) disposed within the handle and intersecting a pathway for the tension component. In some embodiments, the system may further comprise a winder tool (250) configured to be inserted into the receiving area (255) of the handle, and capture and wind the tension component (150). As the tension component (150) is wound onto the winder tool (250), tension is applied to the tension component (150).

According to some embodiments, the present invention features a method of treating a bone fracture between a first bone portion (4a) and a second bone portion (4b). The method may comprise implanting a first implant (110) that is coupled to a flexible tension component (150), via an attachment component (118), into the first bone portion (4a), manipulating the tension component (150) so as to reduce the bone fracture between the first bone portion (4a) and the second bone portion (4b), and implanting at least a portion of a second implant (130) into the second bone portion (4b). When tension is applied to the tension component (150), the second implant (10) engages a compressible locking component (155) of the flexible tension component (150), which causes the compressible locking component (155) to collapse upon itself such that the compressible locking component (155) secures the second implant (130) relative to the first implant.

According to other embodiments, the present invention features a method of anchoring a first bone portion (4a) to a second bone portion (4b). The method may comprise implanting a first implant (110) that is coupled to a tension component (150), via an attachment component (118), into the first bone portion (4a), manipulating the tension component (150) so as to reduce the bone fracture between the first bone portion (4a) and the second bone portion (4b), and implanting at least a portion of a second implant (130) into the second bone portion (4b). The step of implanting may comprise applying tension to the tension component (150) such that the second implant (130) engages a compressible locking component (155) of the tension component (150), which causes the compressible locking component (155) to collapse upon itself and secure the second implant (130) relative to the first implant (110). The second implant (10) engages the tension component (150) so as to hold the first bone portion (4a) and the second bone portion (4b) in place.

In some embodiments, the first bone portion (4a) and the second bone portion (4b) are from the same bone or are in different bones. In other embodiments, the first bone portion (4a) is from one bone and the second bone portion (4b) is from another bone.

In some embodiments, the first implant (110) may be radially symmetric. In other embodiments, the first implant (110) may have exterior threads. The first implant (110) may be configured to be anchored or fixated to an interior of the first bone portion (4a). The first implant (110) may be configured to engage an interior of the first bone portion (4a). The interior of the first bone portion (4a) may be a bone tunnel.

According to some embodiments, the present invention features a bone implant (110) comprising a distal component (111) comprising an attachment component (118), and a proximal component (113) having a threaded exterior surface and a lumen (116). The proximal component (113) may be configured to mate with the distal component (111) such that the attachment component (118) is offset from the lumen.

In some embodiments, the distal component (111) may have a rounded or blunted tip (112). In some embodiments, the proximal component (113) further comprises side vent holes (114). In other embodiments, proximal component (113) further comprises at least two grooves disposed along the length of the lumen. The attachment component (118) may align with the grooves and be offset from a central axis of the lumen to prevent obstruction of the lumen (116).

According to some embodiments, the present invention features a bone implant (110) comprising a first component (111) comprising an attachment component (118), and a second component (113) configured to mate with the first component (111). The second component (113) can have a rounded or blunted tip (112), a threaded exterior surface, and a lumen (116). The attachment component (118) may be offset from a central axis of the lumen to prevent obstruction of the lumen (116).

As used herein, “radially symmetric” refers to having a symmetrical arrangement around a single main axis, so that the implant can be divided into similar halves by any plane that contains the main axis. “Bilateral symmetry” refers to a body that can be divided along a plane that splits the body into right and left sides that are mirror images of each other. Without wishing to limit the present invention, the bone implant (110) is radially symmetric, unlike previous implants that have bilateral symmetry.

According to other embodiments, the present invention features a retaining implant (130) comprising a lumen (136) and at least one opening connected to the lumen. In some embodiments, the lumen (136) tapers from the opening. The tapering of the lumen is configured to compress and lock a sliding knot of a suture. A diameter of the lumen may gradually narrow from the first opening such that a taper angle, α, ranges from about 5-25°.

In other embodiments, the retaining implant (130) may further comprise a V-shaped notch (140) disposed on its distal end (132). The V-shaped notch (140) may be configured to restrain and secure a suture, suture tape, or tissue to a bone. The notch (140) is configured to draw in at least a portion of the suture, suture tape, or tissue into a hole.

In some other embodiments, the retaining implant (130) may have a tapering outer diameter reduced in size at a distal end (132) of the retaining implant. The distal end (132) of the retaining implant may be conical or pointed. The retaining implant may be configured to capture and drive a tissue, a suture, or suture tape into a hole. Without wishing to limit the present invention, the tapered distal end (132) allows for easier insertion of said tissue, suture, or suture tape into said hole.

In other embodiments, the retaining implant (130) may further comprise a threaded surface, barbs, or annular protrusions disposed on its outer surface. In some embodiments, the retaining implant (130) may further comprise a ferrule (138) disposed at the opening.

In other embodiments, the retaining implant (130) may be T-shaped having T-arms (138) disposed at its proximal end (134). In some other embodiments, the retaining implant (130) may be L-shaped having an arm (138) disposed at its proximal end (134). In some embodiments, the arm (138) is configured to link a suture, suture tape, or tissue with another tissue. For instance, the arm (138) may be configured to attach a suture, suture tape, or tissue to a bone surface.

According to some embodiments, the present invention provides a method of anchoring a first tissue portion (4a) to a second tissue portion (4b). The method may comprise implanting a first implant (110) that is coupled to a tension component (150), via an attachment component (118), into the first tissue portion (4a), and implanting at least a portion of a second implant (130) into the second tissue portion (4b). In some embodiments, implanting may comprise applying tension to the tension component (150) such that the second implant (130) engages a compressible locking component (155) of the tension component (150), which causes the compressible locking component (155) to collapse upon itself and secure the second implant (130) relative to the first implant (110). The second implant (10) engages and secures the tension component (150) in place. In other embodiments, the second implant (10) further captures a third tissue portion and couples it to the second tissue portion (4b). In some embodiments, the first tissue portion (4a) and the second tissue portion (4b) may be bone. The third tissue portion may be a ligament or tendon.

According to other embodiments, the present invention provides a driver tool (200) comprising a drive shaft (203) and a handle (205) connected to the drive shaft (203). In some embodiments, the handle (205) has a storage compartment (217) and grooves (215) that lead to said storage compartment (217). The storage compartment (217) may be configured to retain an implant prior to use, and the grooves (215) may be configured to retain at least a portion of a suture (150) prior to use. In other embodiments, the handle (205) may further comprise a receiving area (255) disposed within the handle and that intersects a pathway for the suture. The driver tool (200) may further comprise a winder tool (250) configured to be inserted into the receiving area (255) of the handle, and capture and wind the suture (150). The winder tool (250) may be rod shaped with a slot that captures the suture. Twisting the winder tool (250) causes the suture (150) to be wound onto the winder tool (250), which causes tension to be applied to the suture (150).

According to other embodiments, the present invention features a tissue or fracture fixation system (100) comprising a flexible tension component (150), a first implant (110) comprising an attachment component (118) configured to couple the tension component (150) to the first implant (110), and a second implant (130) configured to engage the tension component (150) so as to secure the first implant (110) relative to the second implant (130). Without wishing to limit the present invention, the tension component (150) allows for fracture reduction of the bone fragment relative to a second bone fragment.

In other embodiments, the tissue or fracture fixation system (100) comprises a flexible tension component (150), a first implant (110) comprising a lumen (116) configured for use with a guidewire and an attachment component (118) configured to couple the tension component (150) with the first implant (110), and a second implant (130) configured to engage the tension component (150) to secure the first implant (110) relative to the second implant (130). The attachment component (118) may be offset from a central axis of the lumen.

In some embodiments, the attachment component (118) couples the tension component (150) to the first implant (110) to allow for cannulation. In some embodiments, the attachment component (118) acts as a drive mechanism that engages the tension component (150). For example, the attachment component (118) acts as a pulley and the tension component (150) slides about the attachment component (118).

In other embodiments, the system may include at least one supporting implant (300) disposed between the first implant (110) and the second implant (130). In one embodiment, a portion of the tension component (150) may be disposed through a lumen (310) of the supporting implant. In another embodiment, the tension component (150) may be adjacent to the supporting implant (300). In some embodiments, the supporting implant (300) may have surface treatments and nanotechnology treatments to stimulate bone growth. In other embodiments, the lumen (310) is treated with one or more anti-clotting, antithrombogenic coatings.

In some other embodiments, the first implant (110) comprises a distal component (111) having a U-shaped catch groove (118) and a proximal component (113) removably coupled to the distal component (111). The proximal component (113) can have a lumen (116) disposed therein for receiving a portion of the distal component (111) in the lumen. In some embodiments, the tension component (150) is configured to be disposed through the lumen (116) of the proximal component (113) and looped around the U-shaped catch groove (118) of the distal component (111).

In other embodiments, the proximal component (113) further comprises grooves (120) disposed along the length of the lumen. In some other embodiments, the ends of the U-shaped catch groove (118) of the distal component may be configured to align with the grooves (120) of the proximal component. Grooves (120) act as pathways for the tension component (150). For example, the grooves (118, 120) act as a pulley mechanism and the tension component (150) slides about the grooves.

In some embodiments, the distal component (111) of the first implant comprises a rounded atraumatic tip. In some embodiments, the proximal component (113) may comprise side vents (114) for sterilization. The vents may be disposed along the length of the proximal component.

In some embodiments, the first implant (110) and/or the second implant (130) has a threaded exterior surface. In some embodiments, the distal component (111) of the first implant (110) has a rounded or blunted tip (112).

In some embodiments, the tension component (150) is a suture. In some embodiments, the suture may be constructed from a polymer material or a metal wire material. In alternative embodiments, the tension component (150) comprises a zip tie tape portion. In some embodiments, the zip tie tape portion may be constructed from a polymer material or a metal.

In some embodiments, the second implant (130) has a locking mechanism for securing the tension component (150). For example, the second implant (130) has a lumen (136) that tapers inwardly from the ends such that the interior diameter of the lumen is smaller than the diameter at the ends of the second implant. The second implant (130) may include a ferrule or rim (138) that can rest against an outer surface of the bone portion when the second implant (130) is inserted into the bone portion. In some embodiments, an end of the tension component (150) may be tied into a slipknot (155) that is large enough such that it cannot pass through the narrow interior portion of the lumen. Thus, pulling the moveable end of the tension component causes the slipknot (155) to slide and press against the lumen (136) of the second implant, which brings the first implant and second implant closer together to reduce the fracture. The bone portions may be held in place by the tension in the tension component.

In some embodiments, the system may further comprise one or more auxiliary implants. These one or more auxiliary implants are configured to be implanted into bone fragments to hold them together. In other embodiments, the one or more auxiliary implants may be configured for use with a fracture plating system. For example, a fracture plate may be attached to a bone fragment using one or more auxiliary implants.

In some embodiments, the first implant (110), the second implant (130), or both may be treated with one or more anti-clotting, antithrombogenic coatings. In other embodiments, the first implant (110), the second implant (130), at least one supporting implant, one or more auxiliary implants, or a combination thereof is treated with one or more anti-clotting, antithrombogenic coatings.

According to some embodiments, the present invention features a method of treating a bone fracture between a first bone portion (4a) and a second bone portion (4b). For example, the method may be used for fracture reduction and bone fixation. IN some embodiments, the method may comprise implanting a first implant (110) that is coupled to a tension component (150), via an attachment component (118), into the first bone portion (4a), manipulating the tension component (150) so as to reduce the bone fracture between the first bone portion (4a) and the second bone portion (4b), implanting at least a portion of a second implant (130) into the second bone portion (4b) with the tension component (150) is disposed through a lumen (136) of the second implant, tying a portion of the tension component (150) to form a knot bundle (155) that can move about the tension component (150), adjusting the tension component (150) such that the knot bundle (155) moves within and presses against the lumen (136) of the second implant, thereby causing tension in the tension component (150) so as to hold the first bone portion (4a) and the second bone portion (4b) in place.

In some embodiments, the first implant (110) is inserted using a driver device (200). In some embodiments, a portion of the flexible tension component (150) can be disposed through a driver tube (210) of the driver device. In other embodiments, the second implant (130) is inserted using the same driver device or alternatively, a different type of driver device.

In some embodiments, the method further comprises creating a first tunnel (7a) in the first bone portion (4a) and a second tunnel (7a) in the second bone portion (4b), prior to implantation of the first implant (110). The first tunnel (7a) and the second tunnel (7b) may be co-axial or approximately co-axial. When the fracture is reduced, the first tunnel (7a) and the second tunnel (7b) are misaligned.

In other embodiments, the method further comprises inserting at least one supporting implant (300) into the first bone portion (4a) and the second bone portion (4b) prior to implanting the second implant (130). In some embodiments, the supporting implant (300) traverses both bone portions and intersects the bone fracture. In other embodiments, the tension component (150) is disposed through a lumen (310) of the supporting implant or adjacent to the supporting implant (300).

In some embodiments, the entire second implant (130) is implanted into the second bone portion (4b). In other embodiments, a majority of the second implant (130) is implanted, and a small section is left out.

In some embodiments, the method may further comprise placing a fracture plate on the first bone portion (4a) and the second bone portion (4b) such that the fracture plate (70) overlaps at least a portion of the bone fracture.

In some other embodiments, the method further comprises inserting one or more auxiliary implants in the first bone portion (4a) and the second bone portion (4b) so as to hold the first bone portion (4a) and the second bone portion (4b) together. Alternatively or in conjunction, the method may further comprise placing a fracture plate on the first bone portion and the second bone portion (4b) such that the fracture plate overlaps at least a portion of the bone fracture. In some embodiments, one or more auxiliary implants may be inserted through the fracture plate and one or both of the bone portions so as to secure the fracture plate the bone portions and hold the bone portions together. In some embodiments, the one or more auxiliary implants can be inserted using an auxiliary implant driver. The one or more auxiliary implants may be inserted at different sections of the bone portions.

In some embodiments, the first implant (110) is a two-part device comprising a distal component (111) having a catch groove (118) and a proximal component (113) having a threaded exterior surface and a lumen (116) disposed therein. In some embodiments, coupling the first implant (110) and the tension component (150) comprises looping the tension component (150) around the catch groove (118) of the distal component (111) and passing it through the lumen (116) of the proximal component (113). A proximal portion of the distal component (111) is then inserted into the distal end of the proximal component (113), said proximal portion including the catch groove (118).

In preferred embodiments, the tension component (150) is of a sufficient length that allows for adjustment and repositioning of the first bone portion. In addition, the tension component (150) is of a sufficient length to form a knot bundle (155) and to allow for tension between the first and second implants. After securing the tension component (150) to the second implant, the length of the tension component may be modified to a desired length, e.g., cut shorter to prevent excess material from coming out of the bone portions.

Non-limiting, alternative embodiments of the present invention may include molded parts, single construction, multi-component constructs, different routing of sutures, other means of securing the first implant relative to the second implant, and/or other configurations of placement of the second implant relative to the first implant.

Further details of the present invention are provided in the following description. It is to be understood that the embodiments described herein are not intended to limit the present invention in any way. Equivalents or substitutes are within the scope of the present invention.

### Implants

In the surgical procedure of the subject device, following placement of guide wire (8), a cannulated drill bit (not shown) is passed over the guide wire to create a pilot hole (FIG. 7). As seen in FIGS. 8A-8C, a first implant (110) is designed such that the most distal portion (111) of the first implant is a separate component, which assembles into a threaded component (113). Both of these components (111, 113) are configured so that they can drill their way into a bone segment.

As shown in the exploded view of FIG. 8A, in some embodiments, the first implant (110) comprises a distal component (111) and a proximal component (113). In some embodiments, the first implant (110) has a square drive, instead of a hex drive, which can reduce the diameter of the first implant (110) by 15%. The tip of the distal component (111) may be rounded or blunted. In other embodiments, holes may be cut or drilled along the length of the proximal portion (113) for venting. These vents may be used for sterilizing the proximal portion (113).

In some embodiments, the first implant (110) has a square drive. In other embodiments, the first implant (110) has a hex or star drive. In some embodiments, this first implant (110) can be configured so that it can be driven with a driver device (200).

In some embodiments, the proximal component (113) comprises a plurality of pathways (120) for the tension component. In some embodiments, the distal component (111) may have a U-shaped groove (118) for receiving a suture. In other embodiments, the distal component (111) can have a second groove. The proximal component (113) may have multiple grooves (120) disposed along the length of its lumen. Two of the grooves (120) may align with the ends of the U-shaped groove (118) to create a continuous pathway for the suture. This is further illustrated in FIG. 8B.

In some embodiments, the distal component grooves allow for a passage of suture (150) through the distal component (111) and serve as a sliding pulley for a limb of the suture or suture tape. When assembled into the proximal component (113) of the first implant, the suture (150) can pass freely through the implant. These grooves are configured to align with the grooves in the proximal component (113) of the first implant, so that the suture (150) has a free passage through the device.

In some embodiments, the suture limbs pass through the groove structures and allow for free movement of the suture. When the distal and proximal components (111, 113) of the first implant are assembled together, the pulley mechanism is created through the first implant and limbs of the suture can move freely through these elements.

Referring to FIG. 8B, the second groove of the distal component may align with a third groove of the proximal component to create another pathway. This pathway allows for the addition of another suture (160) that is static. In some embodiments, the static suture (160) can act as a guide for locating a guide wire, or taking the place of a guide wire, or for screw removal. For example, the static suture (160) can act as a guide for finding the first implant when it is inserted deep in the bone.

Referring to FIG. 8C, the distal component (111) mates with the proximal component (113) to form the assembled first implant. The first implant (110) is configured to provide minimal soft tissue disruption, and to be less palpable for a patient. For instance, the first implant (110) can be made shorter and thinner. The rounded tip enables placement of the implant in the bone cortex to allow for full engagement of threads with minimal protrusion.

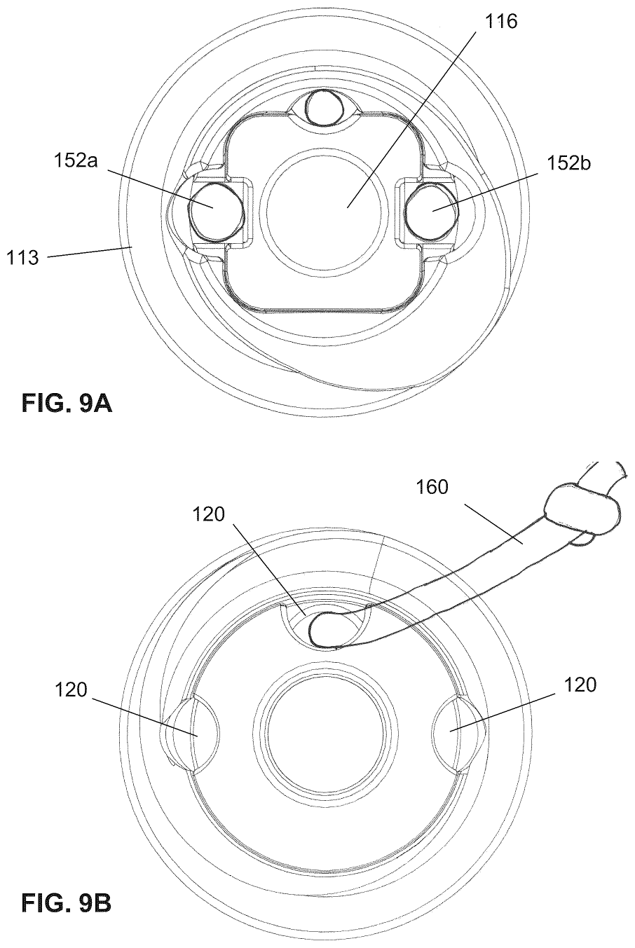

As shown in FIG. 9A, the proximal component (113) can have more than two suture pathways. In some embodiments, a suture is looped around the U-shaped groove (118) and extends through the lumen (116) of the proximal portion, shown on the left and right grooves. In an alternative embodiment, the grooves (120) may be mirrored to create a second pulley mechanism. The distal component (111) may comprise one or more catch grooves (118) to allow for one or more sutures to be coupled to the distal component (111). Each catch groove (118) is paired to a suture (150) and acts as a sliding pulley for said suture. The proximal component (113) may comprise a plurality of grooves (120) to accommodate one suture limb, or alternatively, each groove can accommodate two suture limbs.

As shown in FIG. 9B, the third groove is for the other static suture. The static suture (160) may also be used to retain the front aspect of the distal component, and may also be used in clinical application for removal.

In some embodiments, it is anticipated that a push-in configuration of this device may also utilize the same novel concepts but achieve fixation and like function through means other than bone threads.

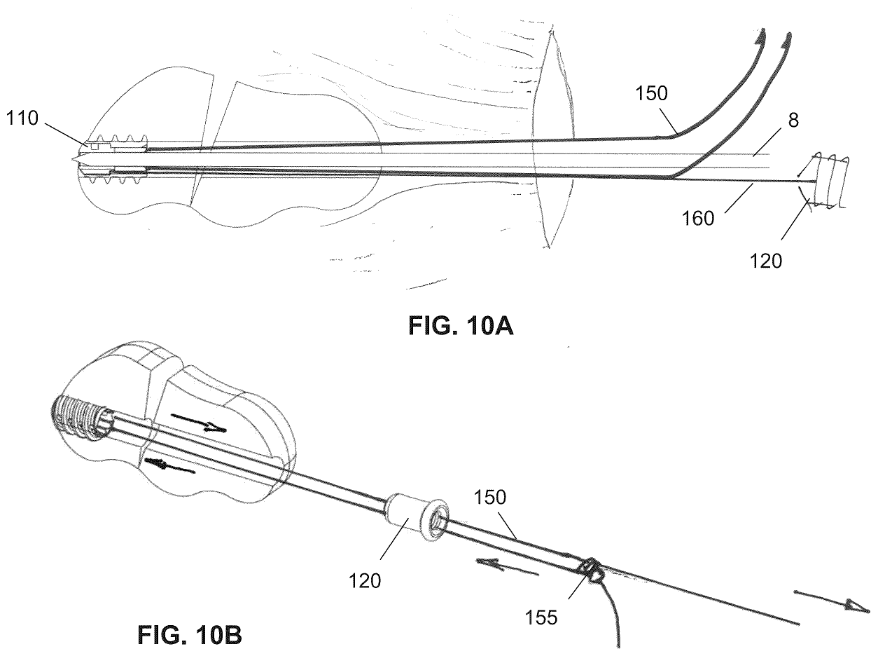

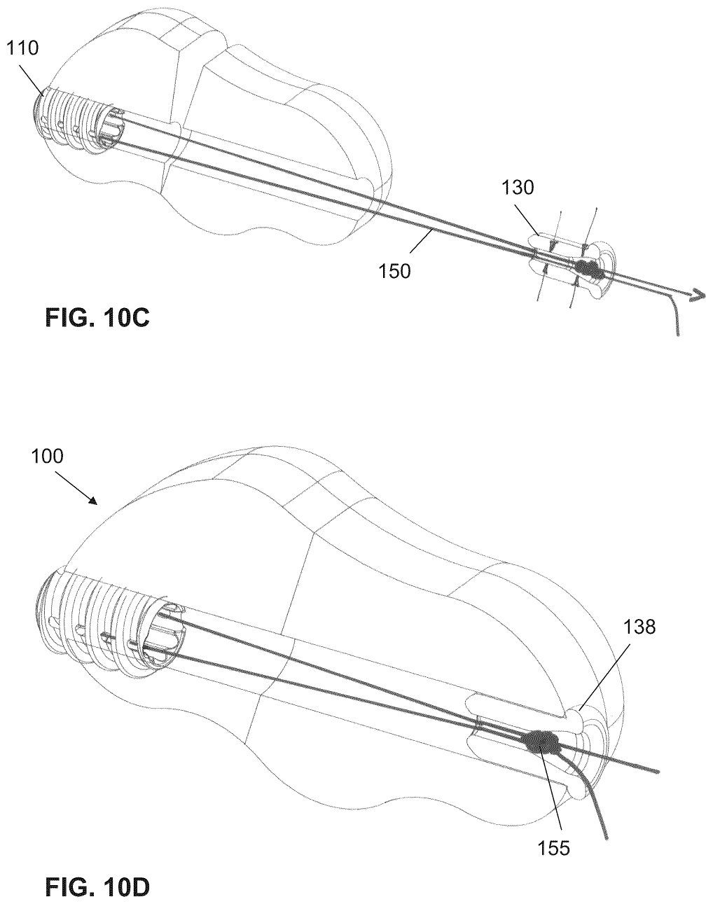

Referring to FIG. 10A, the first implant (110) is inserted into a bone portion. In some embodiments, the first implant (110) is coupled to a static suture (160) and to a suture (150) that is looped around the attachment component (118). When the guide wire is removed, the static suture (160) can be used for orientation and delivery of the next implant. Without wishing to limit the invention to a particular theory or mechanism, the static suture (160) is useful for obstructed views or minimal access surgical incision techniques.

Referring to FIG. 10B, in a non-limiting embodiment, the pulley mechanism is implemented by inserting the ends of the looped suture (150) through the lumen of the second implant (130), and creating a knot bundle (155) by knotting one side of the suture around the untied side of the suture such that the knot bundle (155) can slide about the untied side. The knot bundle (155) has a larger diameter such that it cannot pass through the ferrule (135) of the second implant. The untied side of the suture is pulled in a direction away from the first implant (110), which causes the knot bundle (155) to slide about the untied side and press against the ferrule. The knot bundle (155) drives the ferrule (135) forward and pushes the second implant into a bone portion

Referring to FIG. 10C, the lumen (136) of the second implant has a tapering diameter from the proximal end so that the interior diameter of the lumen is smaller than the diameter at the ferrule. In a non-limiting embodiment, for example, the lumen (136) of the second implant tapers such that it has a ferrule opening diameter of about 0.08 inch, and a pinching diameter of about 0.04 inch in the interior of the lumen. A #2 suture may be used and the sliding knot bundle (155) enters the ferrule opening diameter but is prevented from further advancing into the interior of the lumen by the pinching diameter. This tapering inner diameter compresses knot bundle (155) onto itself, thereby locking down the sliding knot. In some embodiments, a final locking knot is added for strength and security.

FIG. 10D shows the reduction system of the present invention in the reduced fracture configuration. The sliding knot bundle (155) is compressed and not able to travel further in distally. The knot bundle (155) is compressed maximally onto the sliding leg of the suture to lock into position.

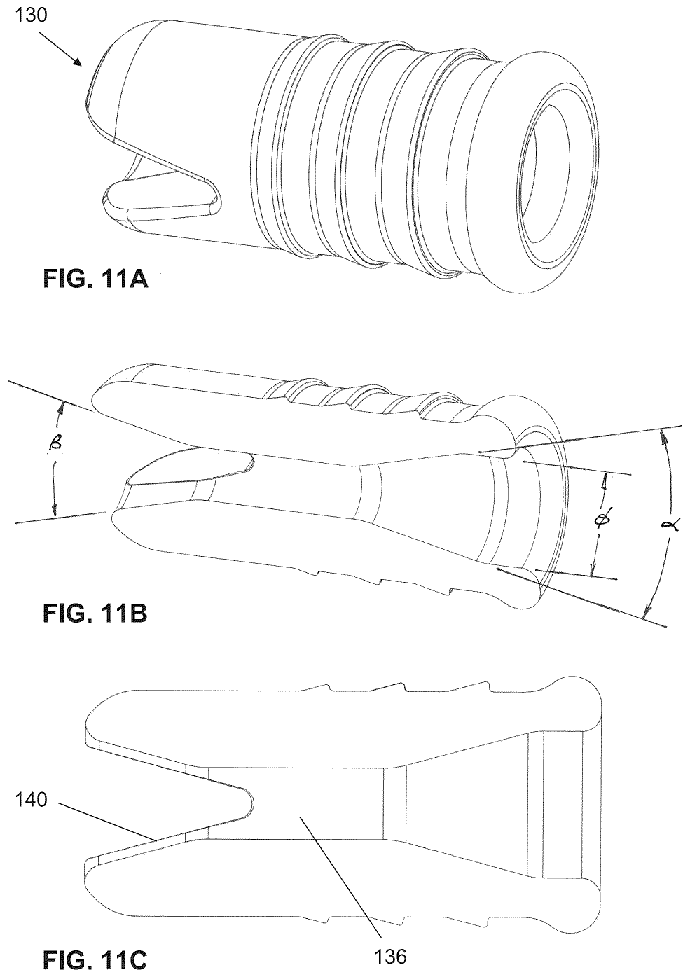

An alternative, non-limiting embodiment of the second implant is shown in FIG. 11A. In some embodiments, the second implant (113) may comprise a longer ferrule with a V-shaped notch (140) at the distal end. Alternatively or in conjunction, the second implant (113) may have barbs or circular protrusions or revolved elements disposed on its outer surface to increase friction and allow for press fitting at the implant site. These elements may improve security of the second implant.

As shown in FIG. 11B-11C, the ferrule can have a tapering inner diameter at both the proximal and distal end. Without wishing to be limited to a particular mechanism, the tapering at both ends allows for multiple clinical uses as sliding and locking the knot can be on the distal end, proximal end, or both. In some embodiments, the diameter gradually narrows such that the taper angle at the proximal end, α, and the taper angle at the distal end, β, is about 15°. The opening diameter may be about 0.08 inch, and the interior diameter is about 0.04 inch.

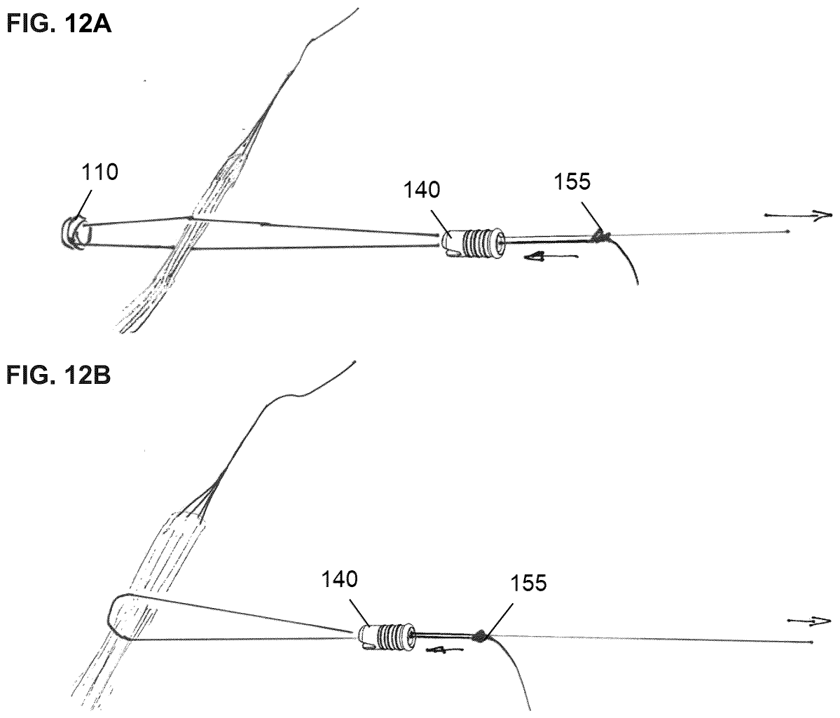

Referring to FIG. 12A-12B, in some embodiments, the V-shaped notch (140) can be utilized as a cinch capture for a suture, suture tape, or tissue (350) when used in conjunction with the first implant (111). By advancing the knot bundle (155) into the proximal end of the second implant, the V-shaped notch (140) catches a tendon, ligament, or suture therein, and said implant can then be fixed into a pre-drilled bone hole or tunnel. The second implant (130) can be inserted into another bone portion that is not axially aligned with the bone portion in which the first implant is inserted, or into a separate bone altogether. For example, the second implant (130) can be anchored to another bone that is adjacent to, but not coaxial with, the fractured bone, thus connecting two different bones together.

When the second implant is used to reattach tendons and ligaments, per FIG. 12A, the V-shaped notch (140) enables insertion of said tissues into the pilot hole of the second bone portion so that the tissues are more closely approximated to the bone tunnel, which is the primary healing site. This method of tissue reattachment is different from attaching or tying down tissue onto the surface of the bone.

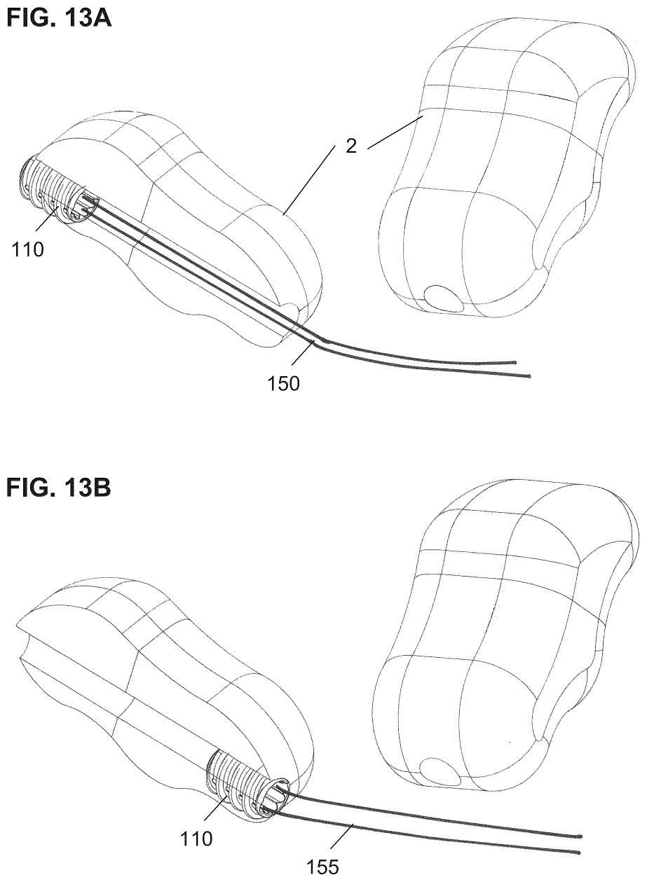

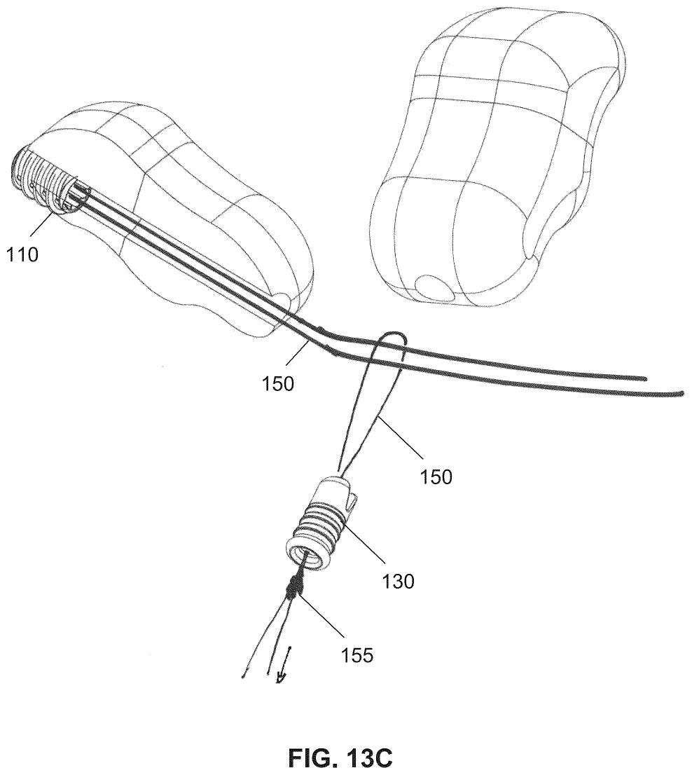

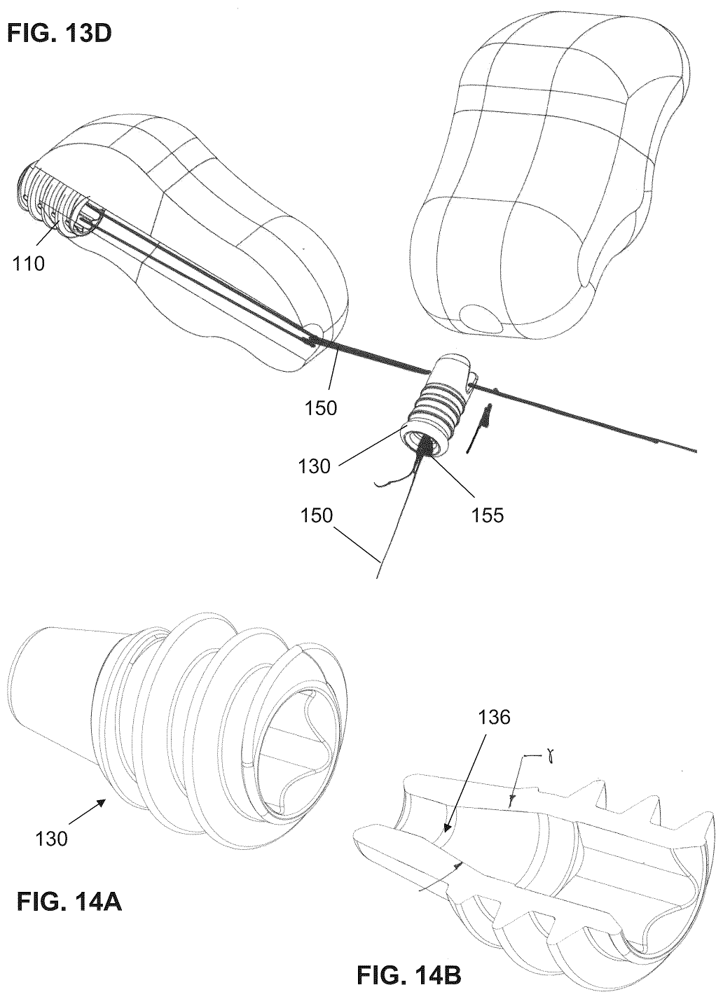

This is in contrast to devices that require the suture or the attached anchoring implant to exit the far side of the bone and be toggled or flipped for the pulley mechanism to work properly. As shown in FIG. 13A-13B, the first implant can be placed so that it engages near (13B) or far (13A) cortex of the bone tunnel. In FIG. 13C, the V-shaped notch (140) captures the suture of the first implant, and another suture is used to secure the first suture to the second implant (130). In FIG. 13D, the second suture has a knot bundle (155) that is slid forward to lock the second suture in place. The second suture can then be inserted into another bone hole or tunnel.

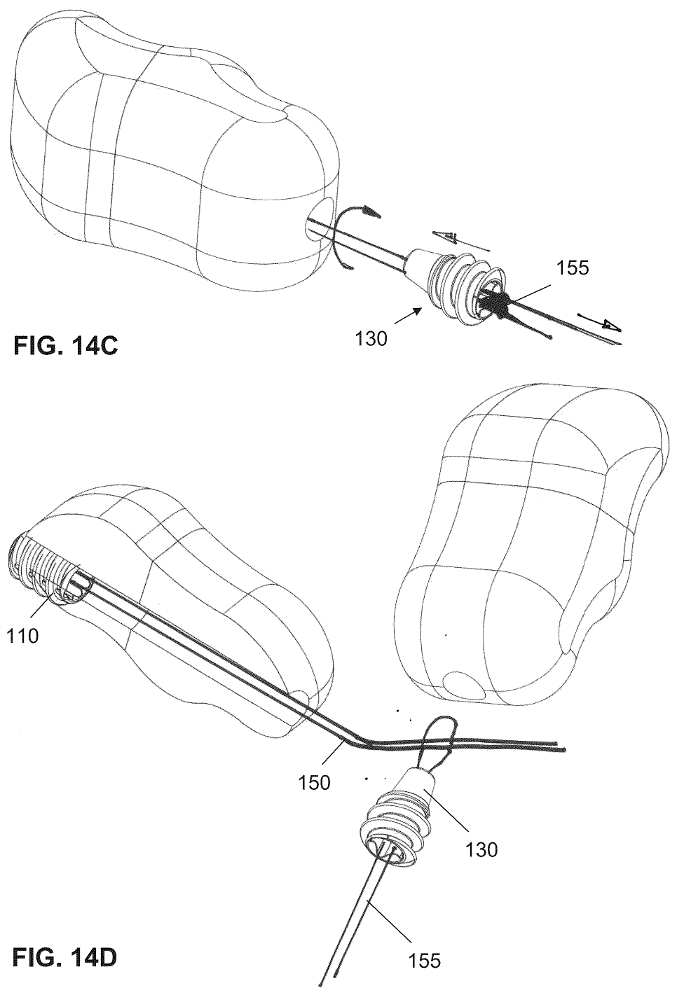

Referring now to FIG. 14A, another embodiment of the second implant (130) has threads to engage the bone tunnel or cortex. As shown in FIG. 14B, this embodiment, like the other ferrule, still has a tapering compression feature from the opening to the interior of the lumen to create the lock function on the sliding knot bundle (155). In FIG. 14C, the threaded ferrule can be driven into the bone tunnel and then the sliding knot bundle (155) pulled into position when used in conjunction with the first implant (110).

Alternatively, in FIG. 14D, the threaded ferrule is used independently in a distinct or separate bone tunnel, or used in conjunction with the first implant in a distinct or separate bone tunnel. This embodiment would allow for settable tension in repairs. A user would be able to tension the adjoined sutures or tissue (350) by pulling the loop end deeper into the construct.

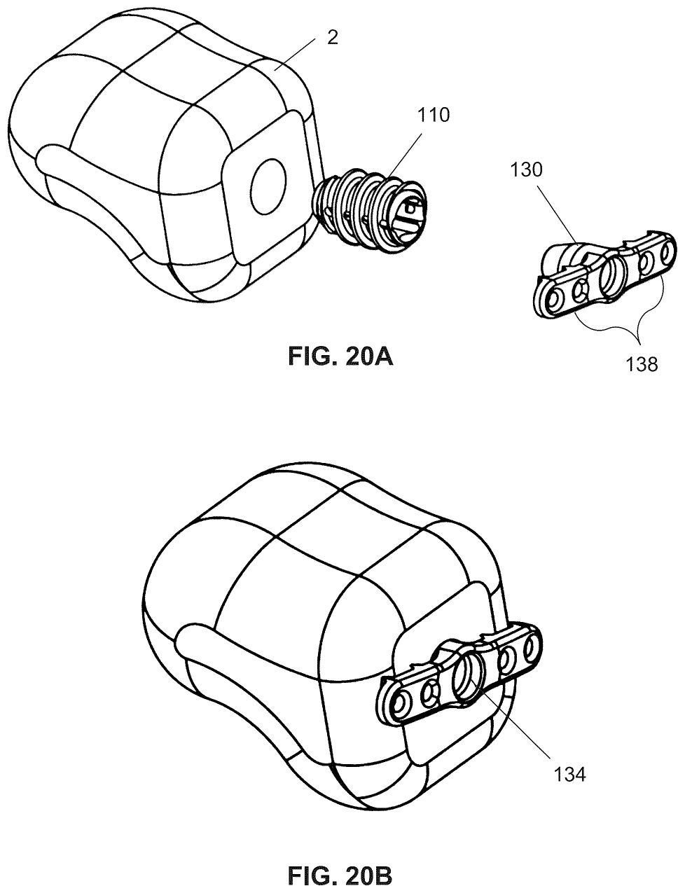

Referring now to FIGS. 20A-22B, another non-limiting embodiment of the second implant (130) features a T-shaped configuration at the proximal end of the second implant. Various configurations of the second implant (130) may feature the T-shaped proximal end combined with an outer surface having ribs or threads, and/or a V-shaped notched at the distal end, and/or tapered opening.

FIGS. 20A-20B shows the second implant (130) having a width aspect such that a “T” configuration (T-shaped) exists. In some embodiments, the second implant may have the same internal tapering lumen (136) to compress the knot bundle (155) as previously described. The implant is configured to enter the pilot hole but then reach a stopping point when the “T” arm (138) makes contact with the bone or tissue. The bone or tissue contacting surface of the implant may be optimized with barbs or sharp features to enhance from on the tissue. Alternatively, the surface may be smooth. The T-arms may also have holes to allow for additional suture or vascular access.

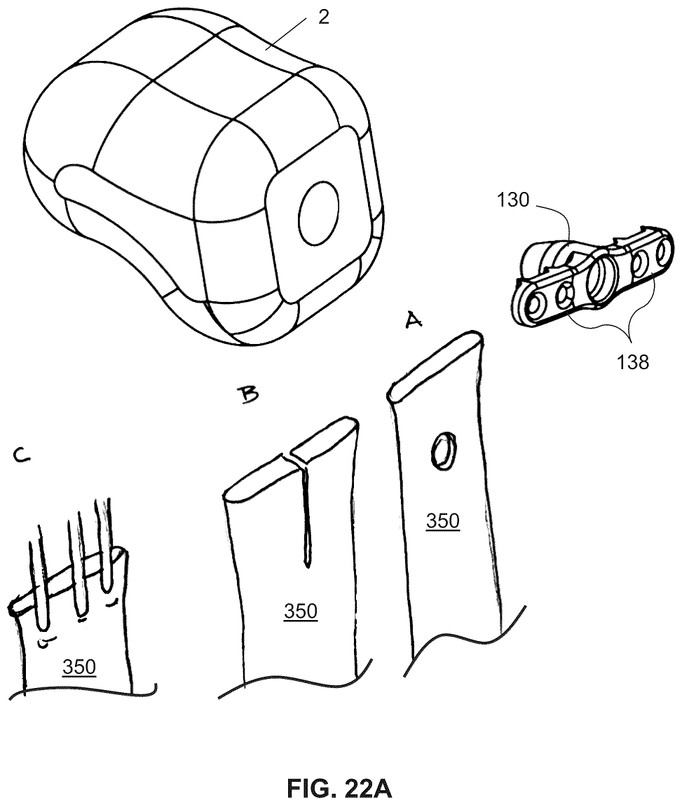

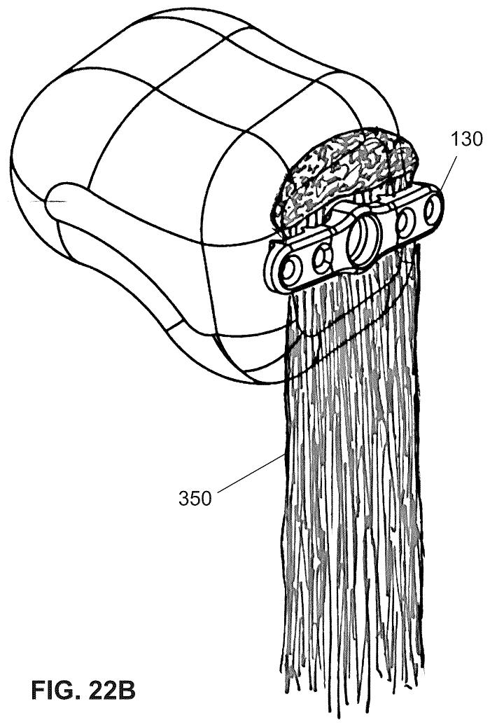

Referring to FIGS. 22A-22B, in some embodiments, the T-shaped second implant (130) can function similarly to other embodiments of the second implant described herein, wherein the tapering inner diameter and the narrowed lumen (136) constrain and compress the sliding knot bundle (155), thus causing the knot (155) to compress on itself and lock in position when the sliding or mobile arm of the suture is tensioned. For instance, as the suture is tensioned, the sliding knot enters the tapered receiving lumen and begins to advance the T-shaped second implant into the pilot hole of the bone. In some embodiments, the taper inner diameter has a pinch angle of about 5-25°. In some embodiments, the narrowed lumen has a diameter of about 0.035-0.04 inches. In other embodiments, the lumen has an opening of about 0.75-0.8 inches.

In some embodiments, the “T” shaped second implant (130) may be used to more broadly distribute tensile loads to the bone and avoid point loads and potential clinical issues, such as stress fractures.

As shown in FIG. 22A, in some embodiments, the T-shaped second implant (130) may be coupled to tissue (350) by placing the first implant thru approximated tissue, creating an access incision in-line with the tissue fibers, and using sutures thru tissue (350) which will be secured to the T-device by clamping, routing, or passing through holes in the T-device. As shown in FIG. 22B, the tissue (350) may be secured to the target site. The tissue may be sandwiched between the T-arms and the bone surface. In some embodiments, a surgeon may opt to decorticate the attachment site to allow recess of the T-shaped second implant (130), or to accelerate or promote tissue to bone healing. In other embodiments, screws may be used to secure the tissue between the T-shaped second implant (130) and the bone. For example, the T-arms may have through holes for receiving screws that anchor the tissue to the bone.

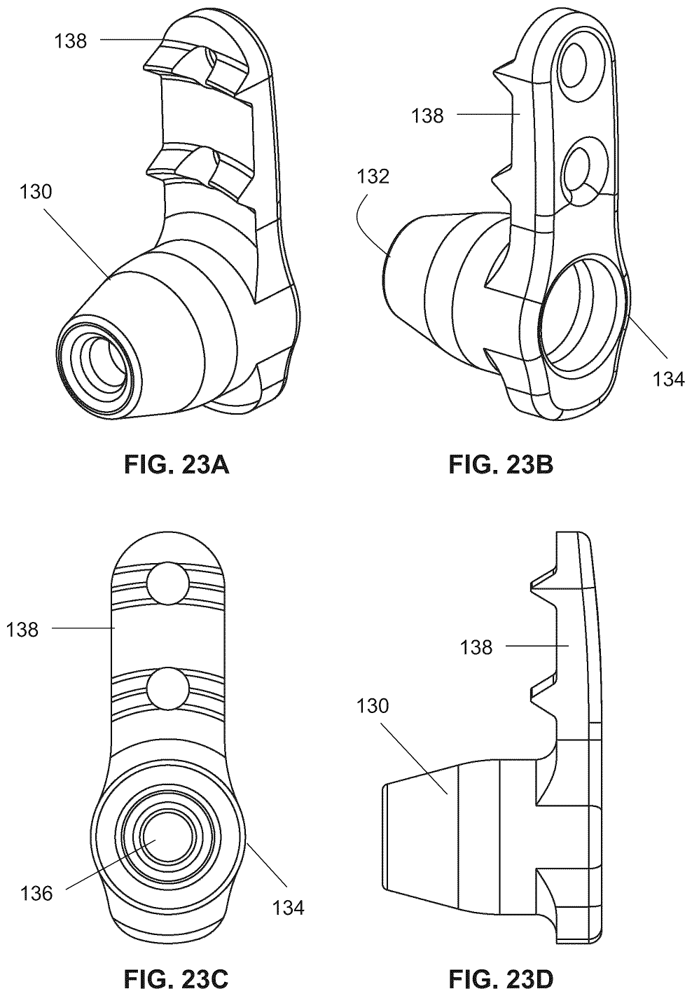

Referring now to FIGS. 23A-23B, an alternative non-limiting embodiment of the second implant (130) features an L-shaped configuration in which a single arm (138) is at the proximal end (134) of the second implant. Various configurations of the second implant (130) may feature the L-shaped proximal end combined with an outer surface having ribs or threads, and/or a V-shaped notched at the distal end, and/or tapered opening. In this embodiment, the second implant may have the same internal tapering lumen (136) to compress the knot bundle (155) as previously described. The implant is intended to enter the pilot hole but then reach a stopping point when the “L” arm (138) makes contact with the bone or tissue. The bone or tissue contacting surface of the implant may be optimized with barbs or sharp features to enhance from on the tissue. Alternatively, the surface may be smooth. The L-arm may also have holes to allow for additional suture or vascular access. In other embodiments, screws may be used to secure the tissue between the L-shaped second implant (130) and the bone.

In some embodiments, the L-shaped second implant (130) can function similarly to other embodiments of the second implant described herein, wherein the tapering inner diameter and the narrowed lumen (136) constrain and compress the sliding knot bundle (155), thus causing the knot (155) to compress on itself and lock in position when the sliding or mobile arm of the suture is tensioned. For instance, as the suture is tensioned, the sliding knot enters the tapered receiving lumen and begins to advance the L-shaped second implant into the pilot hole of the bone. In some embodiments, the taper inner diameter has a pinch angle of about 5-25°. In some embodiments, the narrowed lumen has a diameter of about 0.035-0.04 inches. In other embodiments, the lumen has an opening of about 0.75-0.8 inches.

In some embodiments, the “L” shaped second implant (130) may be used to more broadly distribute tensile loads to the bone and avoid point loads and potential clinical issues, such as stress fractures. In some embodiments, the L-shaped second implant (130) may be coupled to tissue (350) in a manner similar to the T-shaped second implant. For example, the L-arm can have through holes for receiving screws that anchor the tissue to the bone. Without wishing to be bound to a particular mechanism, the L-shaped configuration may be useful when there is a small surface area of bone suitable for connecting to tissue and/or when the second implant is located at or near an edge or corner and/or when the surrounding area is restricted.

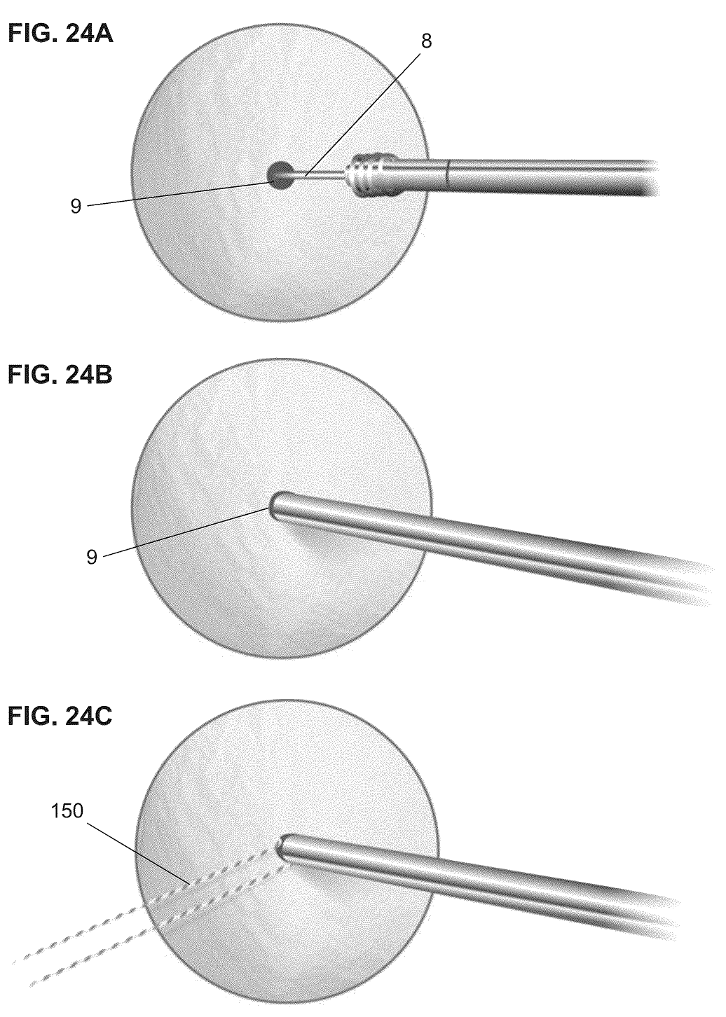

Referring to FIGS. 24A-26B, a non-limiting example of tissue capture and fixation is demonstrated. In FIGS. 24A-24B, a 1.1 mm guide wire is placed for the pilot hole. A 2.75 mm pilot hole is drilled into the bone using a cannulated drill bit. The first implant is driven into position with a driver tool. A minimum depth line may be on the driver tool. In FIG. 24C, the tension suture is released from the driver handle and the driver tool is removed.

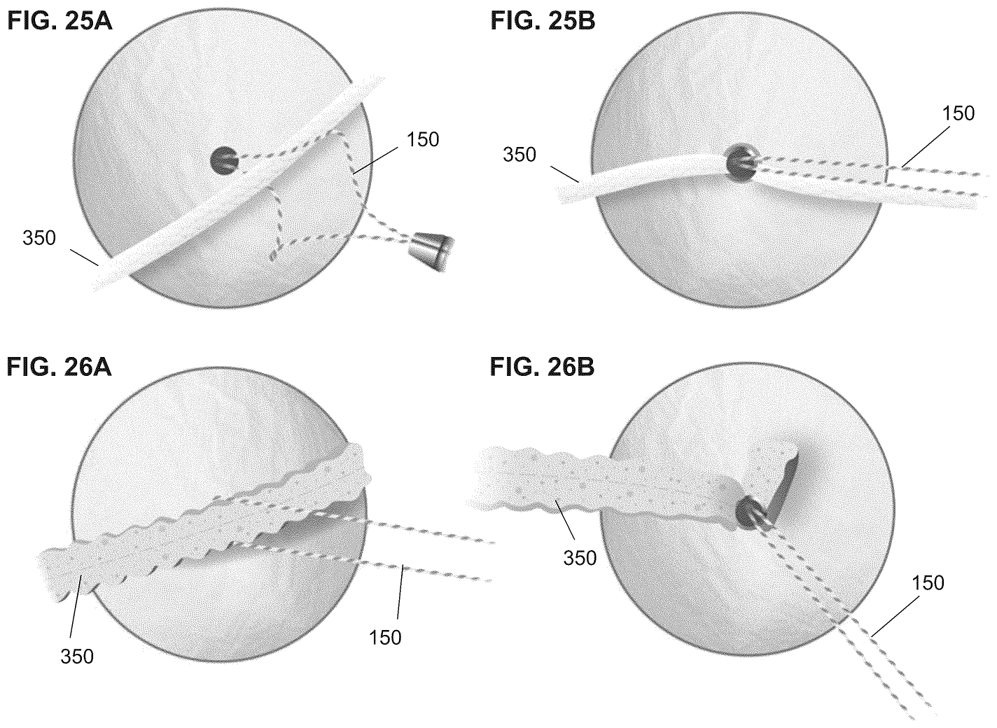

Referring to FIGS. 25A and 26A, a tissue or suture to be secured is positioned between the tension sutures. Referring to FIGS. 25B and 26B, the mobile strand of the tension suture (i.e. the knotted end) is tensioned to advance the pre-tied knot or second implant towards the first implant, capturing and fixating the tissue or suture. A knot pusher is used to fully seat and tighten the pre-tied knot or proximal anchor. In other embodiments, for removal, the implants can be removed by cutting the suture, using the implant driver and/or using a trephine cutter.

In some embodiments, the first and/or second implants may be constructed of a metal, such as a titanium alloy, a stainless steel, or a polymer material, or a combination thereof.

In some embodiments, the first and/or second implants may be treated with one or more anti-clotting, antithrombogenic coatings (Parylene, PTFE, Liquiglide®, Vapor Deposition, Gore, etc.) to create an inner diameter of the implant so that it does not become occluded with blood clot. It is believed that in some bones, including the scaphoid, maintaining a vascular passageway may facilitate healing and continued vascularity of the bony segments.

Without wishing to be bound to a particular theory or mechanism, implants that are abbreviated, blunted or truncated distally may be utilized in thinner or smaller bone fragments or those that may be close to the surface of the skin or other structures.

### Driver

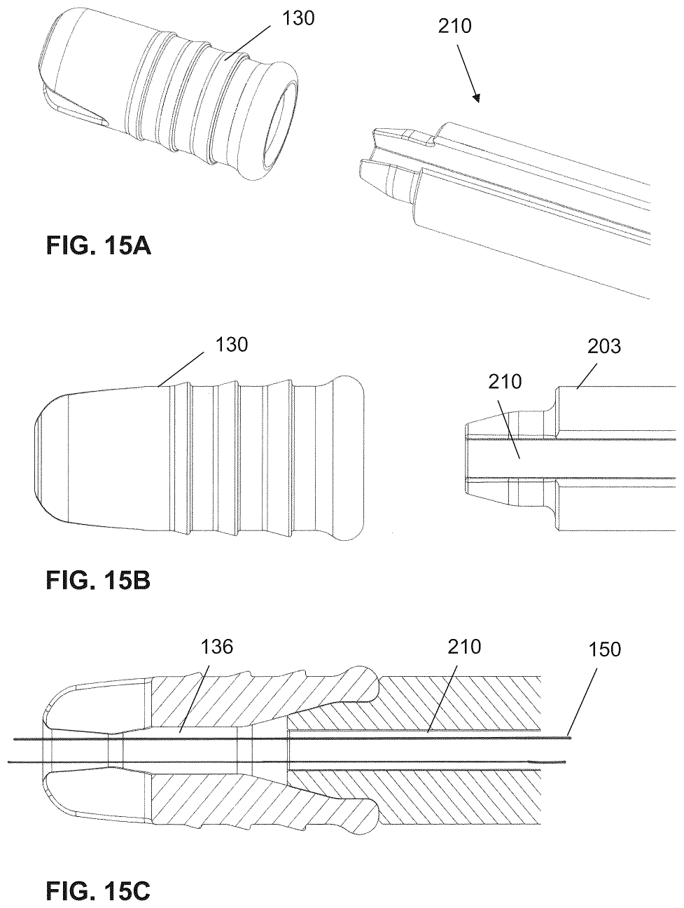

In some embodiments, a driver tool (200) may be used to implant the second implant (130). The distal end of the driver tool is inserted into the proximal opening of the second implant. FIG. 15A-15B shows a driver tool (200) for use with the barbed embodiment of the second implant or even a smooth version. As shown in FIG. 15C, in some embodiments, the distal end of the driver tool is shaped to snuggly fit into the opening of the second implant (130). In some embodiments, the driver tool (200) has an open channel (210), or alternatively a lumen, disposed along its length. The channel (210) can be aligned with the lumen (136) of the second implant. The suture (150) may be disposed through the driver lumen (136) of the second implant and placed inside the channel (210) to prevent tangling. Further distally is the loop or connection to the first implant, and further proximally is the sliding knot bundle (155).

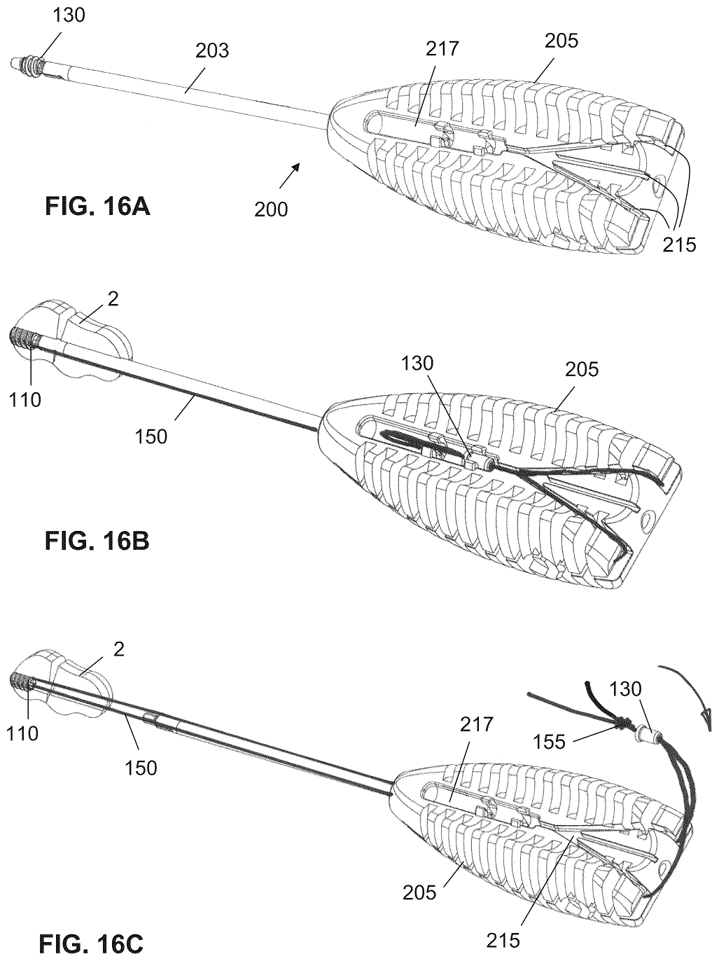

Referring now to FIGS. 16A-16C, another embodiment of the driver tool (200) features a handle (205). The distal end of the driver tool may comprise a star, square, or hex tip. The tip is attached to a driver shaft (203), which is connected to the handle (205). In some embodiments, the driver tool (200) includes multiple suture routings to accommodate multiple product variations and configurations. In some embodiments, the driver tool (200) has a storage compartment (217) for housing the second implant (130) and ends of the suture.

As shown in FIG. 16B, in some embodiments, the handle (205) has grooves (215) disposed on its sides and/or top and/or bottom surfaces that lead to the storage compartment (217). The grooves (215) are configured to route the suture to the storage compartment (217). In some embodiments, the second implant (130) can be retained in the storage compartment (217) while using the driver tool to implant the first implant (110). The second implant (130) can rotate with delivery of the first implant (110), which prevents suture fouling.

In some embodiments, the second implant (130) may face rearward for loading and tensioning. In FIG. 16B, the suture (150) is connected to the first implant (110) and extends along the length of the driver shaft (203); the suture (150) is routed by the handle grooves (215) so that it flips over the back end of the handle and onto the top surface which has the storage compartment (217); the suture is disposed through the second implant (130) that is retained in the storage compartment (217) and terminates in a knot bundle (155), which is also placed in the storage compartment (217). As shown in FIG. 16C, by placing the second implant (130) rearward in the handle (205), it will be oriented correctly when it is released so that the sliding knot (155) is pre-tied and proximal to the second implant (130).

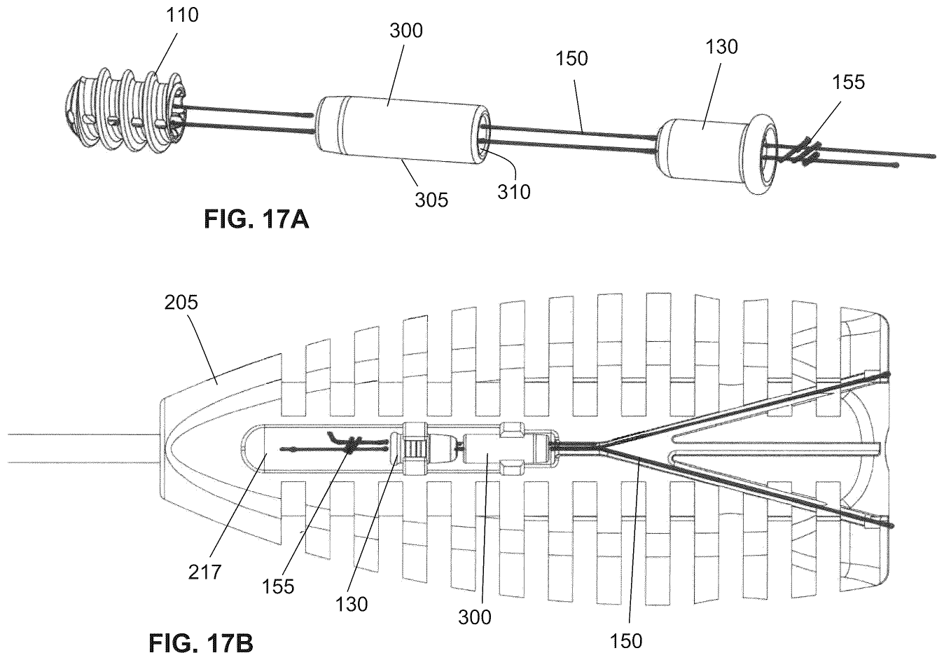

Multiple mounting configurations are possible such that any desired sequence of implants can be delivered in the same fashion. For example, FIG. 17A shows an alternative embodiment in which a support implant (300) is used. The support implant (300) is placed between the first and second implants. FIG. 17B shows the support implant (300) and second implant (130) retained in the storage compartment (217) of the handle.

In some embodiments, the supporting implant can be treated with one or more anti-clotting, antithrombogenic coatings (Parylene, PTFE, Liquiglide®, Vapor Deposition, Gore, etc.) to create an inner diameter of the implant so that it does not become occluded with blood clot. It is believed that in some bones, including the scaphoid, maintaining a vascular passageway may facilitate healing and continued vascularity of the bony segments.

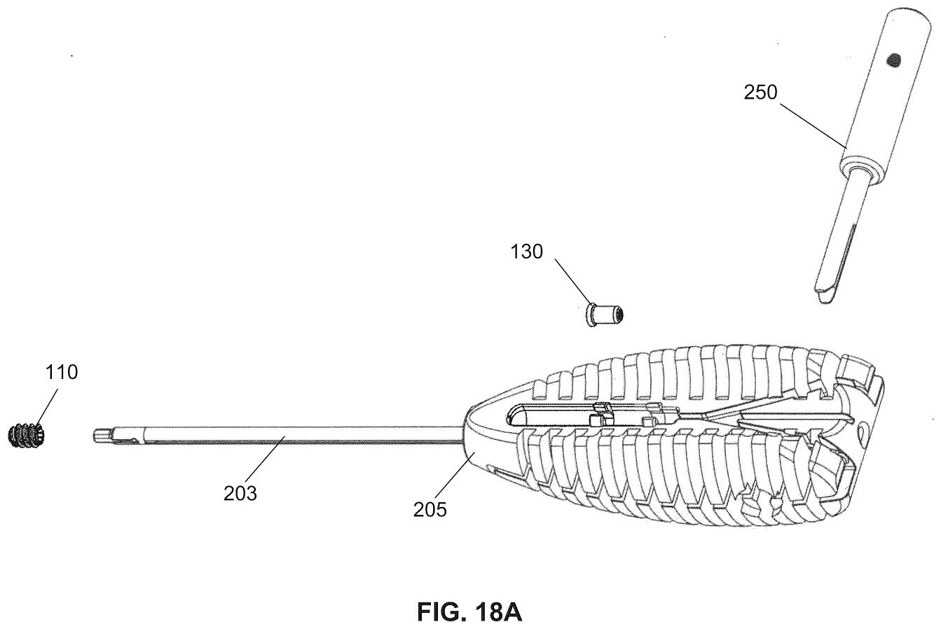

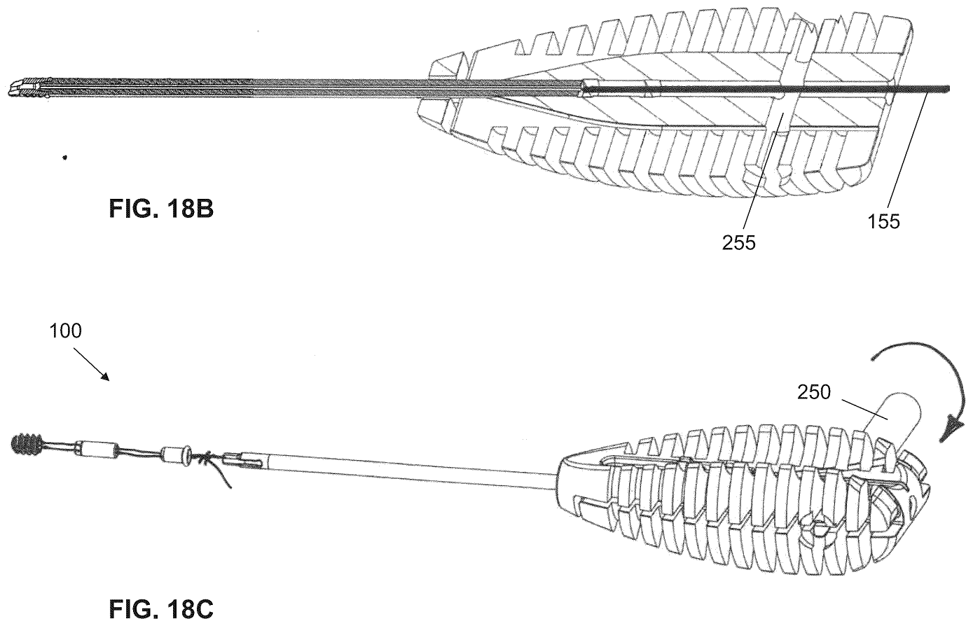

According to another embodiment, in FIG. 18A, the handle is also configured to be used with a winder (250), which is configured to be inserted into a receiving area (255) in the handle body such that it intercepts one or more suture legs. As shown in FIG. 18B, in some embodiments, the suture is disposed along the driver shaft (203) and within a lumen in the handle body, said handle lumen intersecting the receiving area (255). In FIG. 18C, the winder (250) is inserted into the receiving area (255) to catch and capture the suture. The winder (250) is then turned so as to pull on the suture and spool it upon the winder. Without wishing to limit the present invention, the winder (250) provides a mechanical advantage by increasing the tension on the suture and further compressing the sliding knot (155).

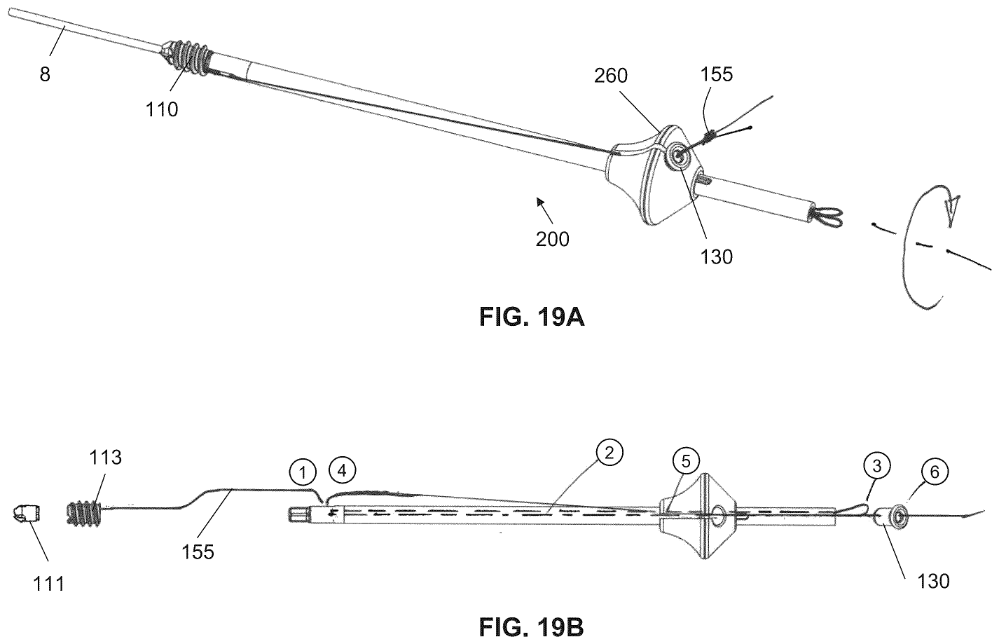

FIG. 19A-19B shows another embodiment of a driver tool (200) for implanting the two-implant reduction system. In some embodiments, the two-implant system is intended to be driven under power with a power drill handpiece. The suture routing allows the first implant (110) to be drilled into place without suture wrap up or tangling around the shaft. In some embodiments, the suture routing allows for the system to be pre-threaded with a sliding knot (155) tied at the manufacturers. FIG. 19B demonstrates the suture routing in the driver tool (200) with only one suture leg shown for clarity. The suture route is as follows:
1—the suture enters distal tip of hex drive;
2—the suture runs the full length of inner shaft;
3—the suture doubles back on itself internal to shaft;
4—the suture exits the distal tip of hex driver;
5—the suture enters a slot in proximal ferrule; and
6—the suture passes through the slot and thru the second implant (130).

In some embodiments, the systems, devices, and/or components described herein can be available in a single-use sterile configuration with a one size fits all or most approach to minimize inventory requirements.

Without wishing to limit the present invention to a particular theory or mechanism, the systems and methods described herein provides reduction system that anchors implants into a segment of bone and allows for a reduction of the fracture without a rigid axial constraint, so that the bone fragments can achieve a best fit under a tensile load. This method can be performed over relatively large distances, using minimal access techniques, and potentially used in conjunction with traditional open reduction and internal fixation (ORIF) techniques and implants.

### Alternative Embodiments

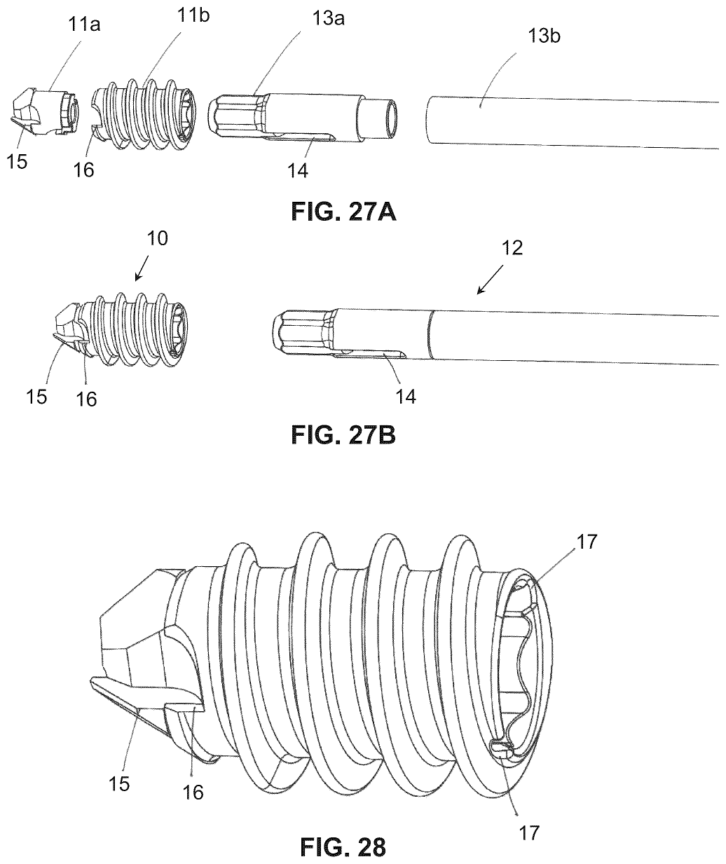

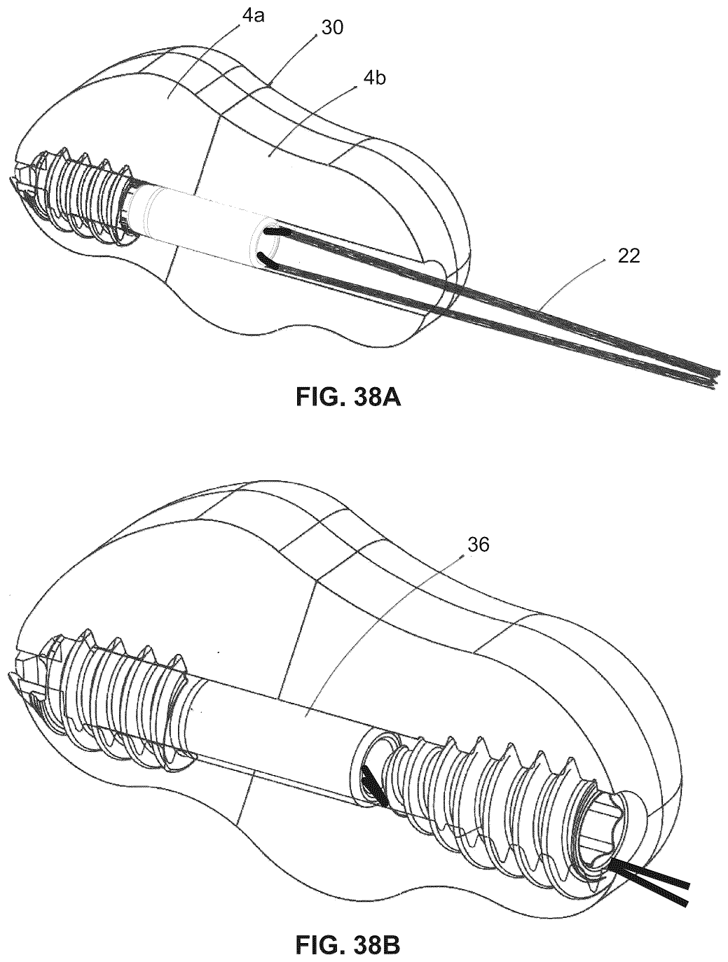

Alternatively or in conjunction with the systems and methods described herein, the present invention provides other embodiments of tissue or fracture fixation systems as shown in FIGS. 27A-50. According to some embodiments, the tissue or fracture fixation system may comprise a flexible tension component (22), a first implant (10) comprising an attachment component (18) configured to couple the tension component (22) to the first implant (10), and a second implant (33) configured to engage the tension component (22) so as to secure the first implant (10) relative to the second implant (33). Without wishing to limit the present invention, the tension component (22) allows for fracture reduction of the bone fragment relative to a second bone fragment. In some embodiments, the attachment component (18) couples the tension component (22) to the first implant (10) to allow for cannulation. In some embodiments, the attachment component (18) acts as a drive mechanism that engages the tension component (22).

In other embodiments, the system may include at least one supporting implant (36) disposed between the first implant (10) and the second implant (33). In one embodiment, a portion of the tension component (22) may be disposed through a lumen (38) of the supporting implant. In another embodiment, the tension component (22) may be adjacent to the supporting implant (36). In some embodiments, the supporting implant (36) may have surface treatments and nanotechnology treatments to stimulate bone growth. In other embodiments, the lumen (38) is treated with one or more anti-clotting, antithrombogenic coatings.

In some embodiments, the first implant (10) has a cutting edge (15). In other embodiments, the second implant (33) has a cutting edge and thread tap (16). In some embodiments, the first implant (10) and/or the second implant (33) is a compression screw. In some embodiments, the first implant (10) and/or the second implant (33) has a threaded exterior surface.

In some other embodiments, the first implant (10) comprises a distal component (11a) having at least one catch groove (18) and a proximal component (11b) removably coupled to the distal component (11a). The proximal component (11b) can have a lumen (21) disposed therein for receiving a portion of the distal component (11a) in the lumen. In some embodiments, the tension component (22) is configured to be disposed through the lumen (21) of the proximal component (11b) and loop around the catch groove (18) of the distal component (11a).

In other embodiments, the distal component (11a) further comprises slide grooves (19, 20) extending from the catch groove (18). In some other embodiments, the proximal component (11b) further comprises grooves (17) disposed in the lumen. The slide grooves (19, 20) of the distal component may be configured to align with the grooves (17) of the proximal component.

In some embodiments, the tension component (22) is a suture. In some embodiments, the suture may be constructed from a polymer material or a metal wire material. In other embodiments, the tension component (22) comprises a zip tie tape portion. The zip tie tape portion may comprise a ratchet track. In some embodiments, the zip tie tape portion may be constructed from a polymer material or a metal.

In one embodiment, the second implant (33) comprises a locking element (63) for securing the tension component (22). For example, the tension component (22) may be a suture that is tied to the locking element (63), such as a loop or bar. In another embodiment, the second implant (33) may comprise a ratcheting lever mechanism that engages the ratchet track of the zip tie tape portion. In some other embodiments, the second implant (33) comprises a boss (59) disposed at a distal end of the second implant. In other embodiments, the second implant (33) comprises a flange (56) disposed at a proximal end of the second implant. In some other embodiments, the second implant (33) has a tapering, bulbous, or flat exterior surface. In some embodiments, the second implant (33) is configured for use with a fracture plating system. For example, a fracture plate (70) may be attached to a bone fragment using the second implant (33).

In some embodiments, the system may further comprise one or more auxiliary implants (40). These one or more auxiliary implants (40) are configured to be implanted into bone fragments to hold them together. In other embodiments, the one or more auxiliary implants (40) may be configured for use with a fracture plating system. For example, a fracture plate (70) may be attached to a bone fragment using one or more auxiliary implants (40).

In some embodiments, the first implant (10), the second implant (33), or both may be treated with one or more anti-clotting, antithrombogenic coatings. In other embodiments, the first implant (10), the second implant (33), at least one supporting implant, one or more auxiliary implants (40), or a combination thereof is treated with one or more anti-clotting, antithrombogenic coatings.

In other embodiments, the tissue or fracture fixation system of the present invention may comprise a first implant (10), at least one elongated, flexible tension component (22) attached and extending from the first implant (10), the tension component (22) having a plurality of grooves, and a second implant (33) comprising at least one through slot (54) for receiving a terminal end of the tension component (22), and a capture mechanism (55) for engaging a groove of the plurality of grooves. In some embodiments, the capture mechanism (55) comprises a ratcheting lever that catches a groove of the plurality of grooves.

According to some embodiments, the present invention features a method of treating a bone fracture between a first bone portion (4a) and a second bone portion (4b). For example, the method may be used for fracture reduction and bone fixation. The method may comprise implanting a first implant (10) that is coupled to a tension component (22), via an attachment component (18), into the first bone portion (4a), manipulating the flexible tension component (22) so as to reduce the bone fracture between the first bone portion (4a) and the second bone portion (4b), and implanting at a portion of a second implant (33) in the second bone portion (4b) such that the second implant (10) engages the flexible tension component (22) so as to hold the first bone portion (4a) and the second bone portion (4b) in place.

In some embodiments, the first implant (10) is inserted using a driver device (12). A portion of the flexible tension component (22) can be disposed through a driver tube (10) of the driver device (12). In other embodiments, the second implant (33) is inserted using the driver device (12) or alternatively, a different type of driver device.

In some embodiments, the method further comprises creating a first channel (7a) in the first bone portion (4a) and a second channel (7a) in the second bone portion (4b), prior to implantation of the first implant (10). The first channel (7a) and the second channel (7b) may be co-axial or approximately co-axial. When the fracture is reduced, the first channel (7a) and the second channel (7b) are misaligned.

In other embodiments, the method further comprises inserting at least one supporting implant (36) into the first bone portion (4a) and the second bone portion (4b) prior to implanting the second implant (33). In some embodiments, the supporting implant (36) traverses both bone portions and intersects the bone fracture. In other embodiments, the tension component (22) is disposed through a lumen (38) of the supporting implant or adjacent to the supporting implant (36).

In some embodiments, the entire second implant (33) is implanted into the second bone portion (4b). In other embodiments, a majority of the second implant (33) is implanted, and a small section is left out. In this case, the small section of the second implant may be used to hold a fracture plate (70). Thus, in some embodiments, the method may further comprise placing a fracture plate (70) on the first bone portion (4a) and the second bone portion (4b) such that the fracture plate (70) overlaps at least a portion of the bone fracture. This placement step is done prior to implantation of the second implant (33). The second implant (33) is then inserted through the fracture plate (70) and into the second bone portion (4b) such that the second implant (33) secures the fracture plate (70) to the second bone portion (4b).

In some other embodiments, the method further comprises inserting one or more auxiliary implants (40) in the first bone portion (4a) and the second bone portion (4b) so as to hold the first bone portion (4a) and the second bone portion (4b) together. Alternatively or in conjunction, the method may further comprise placing a fracture plate (70) on the first bone portion (4a) and the second bone portion (4b) such that the fracture plate (70) overlaps at least a portion of the bone fracture, and inserting one or more auxiliary implants (40) through the fracture plate (70) and one or both of the bone portions so as to secure the fracture plate (70) the bone portions and hold the bone portions together. In some embodiments, the one or more auxiliary implants (40) can be inserted using an auxiliary implant driver (44). The one or more auxiliary implants (40) may be inserted at different sections of the bone portions.

According to other embodiments, the method of the present invention may comprise creating a first channel (7a) in the first bone portion (4a) and a second channel (7a) in the second bone portion (4b), coupling an elongated, flexible tension component (22) to a first implant (10), inserting the first implant (10) and the flexible tension component (22) through the second channel (7b) of the second bone portion (4b) and through the first channel (7a) of the first bone portion (4a) such that the first implant (10) is fixed to the first bone portion (4a) and a portion of the flexible tension component (22) is disposed through the second channel (7b), manipulating the flexible tension component (22) so as to position the first bone portion (4a) and the second bone portion (4b) in a reduced fracture configuration in which a gap or displacement between the first bone portion (4a) and the second bone portion (4b) is reduced, and inserting a second implant (33) through the second channel (7b) such that the second implant (10) is fixed to the second bone portion (4b) and the second implant (10) further engages the flexible tension component (22), which causes tension in the flexible tension component (22) that holds the first bone portion (4a) and the second bone portion (4b) in the reduced fracture configuration.

In some embodiments, the first implant (10) is a two-part device comprising a distal component (11a) having a catch groove (18) and a proximal component (11b) having a threaded exterior surface and a lumen (21) disposed therein. In some embodiments, coupling the first implant (10) and the tension component (22) comprises looping the tension component (22) around the catch groove (18) of the distal component (11a) and passing it through the lumen (21) of the proximal component (11b). A portion of the distal component (11a) is then inserted into the lumen (21) of the proximal component (11b), said portion including the catch groove (18).

According to yet other embodiments, the method of the present invention may comprise creating a first channel (7a) in the first bone portion (4a) and a second channel (7a) in the second bone portion (4b), inserting a first implant (10) having at least one elongated, flexible tension component (22) extending therefrom through the second channel (7b) of the second bone portion (4b) and through the first channel (7a) of the first bone portion (4a) such that the first implant (10) is fixed to the first bone portion (4a) and a portion of the flexible tension component (22) is disposed through the second channel (7b), manipulating the flexible tension component (22) so as to position the first bone portion (4a) and the second bone portion (4b) in a reduced fracture configuration in which a gap or displacement between the first bone portion (4a) and the second bone portion (4b) is reduced, and inserting the tension component (22) through a receiving slot (54) of a second implant (33) until at least a portion of the second implant (33) is disposed in the second channel (7b) so as to cause tension in the flexible tension component (22) that holds the first bone portion (4a) and the second bone portion (4b) in the reduced fracture configuration, and a capture mechanism (55) of the second implant (33) engages a groove of a plurality of grooves of the tension component (22) to lock the tension component (22) in place.

In preferred embodiments, the tension component (22) is of a sufficient length that allows for adjustment and repositioning of the first bone portion. In addition, the tension component (22) is of a sufficient length to create tension between the first and second implants. After securing the tension component (22) to the second implant, the length of the tension component may be modified to a desired length, e.g., cut shorter to prevent excess material from coming out of the bone portions.

Non-limiting, alternative embodiments of the present invention may include molded parts, single construction, multi-component constructs, different routing of sutures, and other means of securing the first implant relative to the second implant.

Further details of the present invention are provided in the following description. It is to be understood that the embodiments described herein are not intended to limit the present invention in any way. Equivalents or substitutes are within the scope of the present invention.

In the surgical procedure of the subject device, following placement of guide wire (8), a cannulated drill bit (not shown) is passed over the guide wire to create a pilot hole (FIG. 7). As seen in FIGS. 27A-27B, a first implant (10) is designed such that the most distal portion (11a) of the first implant is a separate component, which assembles into a threaded component (11b). Both of these components (11a, 11b) are configured so that they can functionally drill and tap their way into a bone segment.

In some embodiments, this first implant (10) can be configured so that it can be driven with a driver device (12) whereby the distal element (13a) of the driver has one or more access slots (14) that allow for a suture component to be passed through and into the longer tube (13b). The driver assembly is joined such that the distal element (13a) and tube (13b) act as a single component.

Referring now to FIG. 28, the assembled first implant (10) has the cutting edge (15) and the cutting and thread tap cutting features (16) that allow the driver to advance the first implant (10) into bone. The proximal component (11b) also has grooves (17) in the lumen, which allow for suture passage along the sides, and separate from the torsional drive feature.

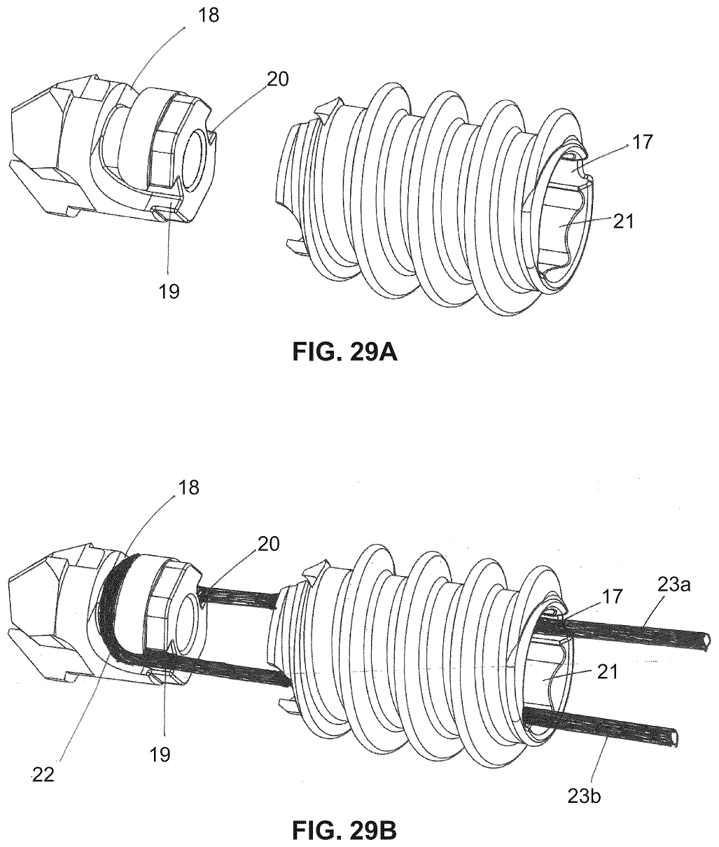

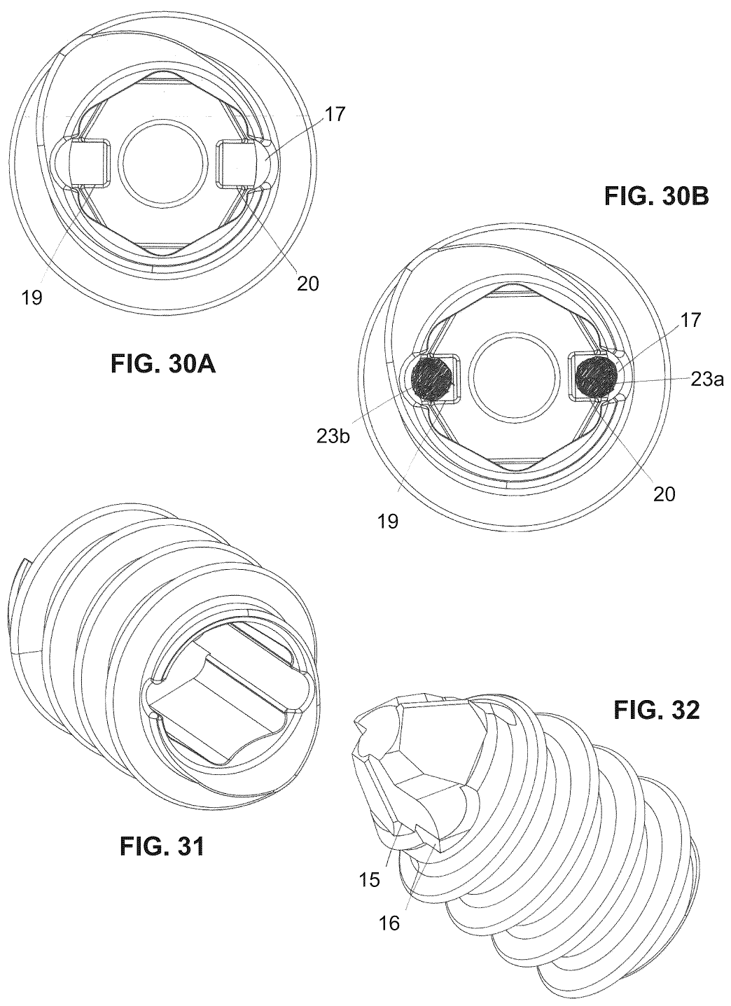

As shown in FIG. 29A, the distal component grooves (18, 19, 20) allows for a passage of suture (22) through the distal component (11a) and serves as a sliding pulley for a limb of the suture or suture tape (22). When assembled into the proximal component (11b) of the first implant, the suture (22) can pass freely through the implant. These grooves (18, 19, 20) are configured to align with the grooves (17) in the proximal portion (11b) of the first implant, so that the suture (22) has a free passage through the device when placed on the delivery driver (12) or delivery instrument. FIG. 29B shows suture limbs (23a, 23b) that pass through the groove structures and allows for free movement of the suture. FIGS. 30A and 30B shows rear views of when the distal and proximal components (11a, 11b) of the first implant are assembled together. The passageway (17) is created through the first implant and limbs of the suture (23a, 23b) can move freely through these elements.

In alternative embodiments, the distal component (11a) may comprise one or more catch grooves to allow for one or more sutures to be coupled to the distal component (11a). Each catch groove (18) is paired to a suture (22) and acts as a sliding pulley for said suture. The proximal component (11b) may comprise a plurality of grooves (17) to accommodate one suture limb, or alternatively, each groove can accommodate two suture limbs.

In some embodiments, it is anticipated that a push-in configuration of this device may also utilize the same novel concepts but achieve fixation and like function through means other than bone threads.

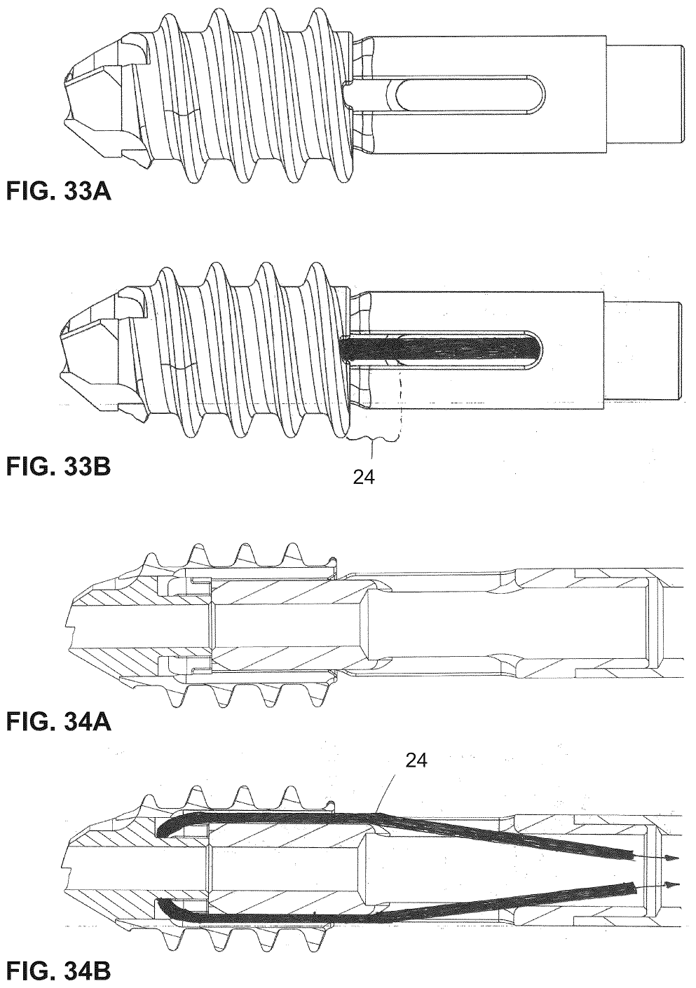

FIG. 33A shows the first implant (10) mounted on its delivery driver (12). In FIG. 33B, the suture (22) passes through the access slots (14) and into the inner lumen of the driver after a short traverse (24). FIGS. 34A-34B shows a cross-sectional view of the first implant (10) mounted on the delivery driver (12) and the suture (22) looped around the catch groove 18, disposed in the grooves (19, 20, 17), and passing through the access slots (14) and into the lumen of the driver tube.

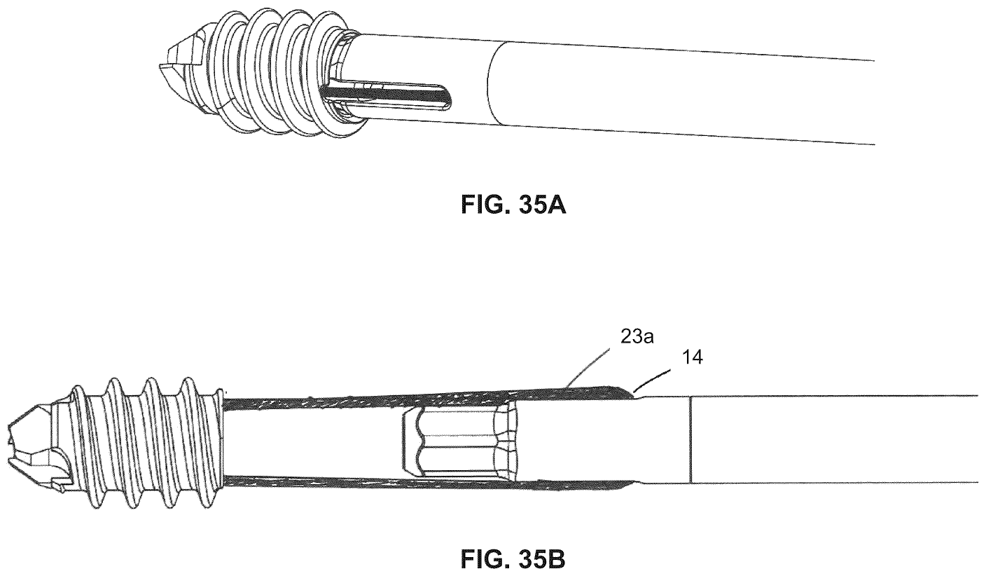

FIG. 35A is another view of the first implant (10) mounted on its delivery driver (12). FIG. 35B illustrates the delivery driver being removed axially from the first implant (10). A suture (22) passes along the access feature (14) in the driver and into the protective tube (13b).

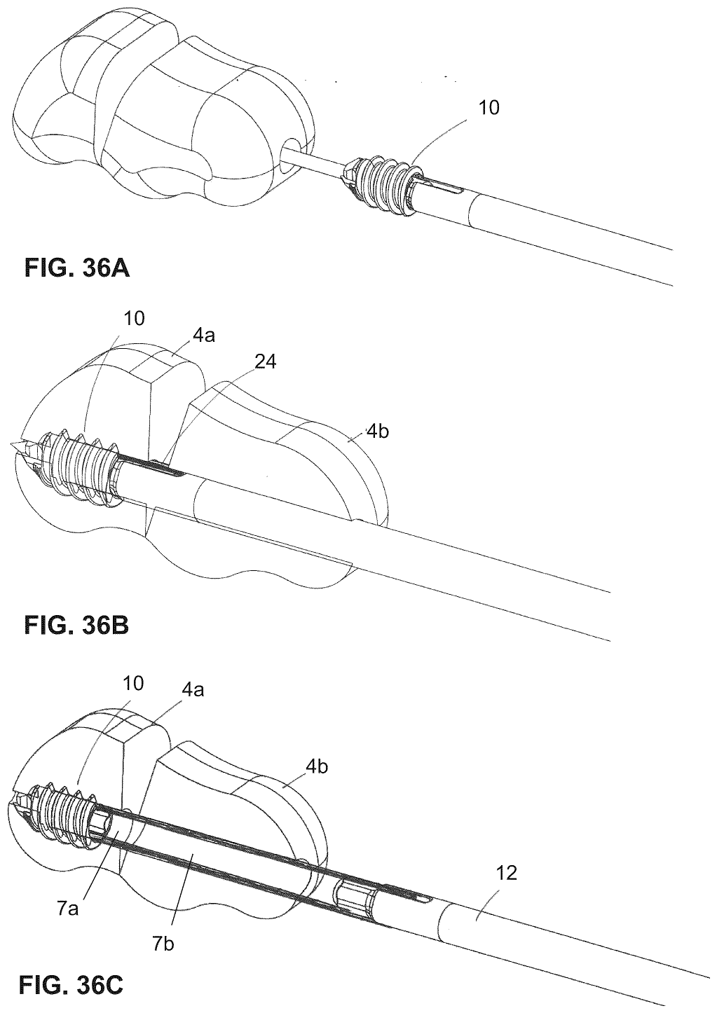

In a non-limiting embodiment, FIG. 36A illustrates the guide wire (8) driven into the scaphoid fragments (4a, 4b), a pilot hole (9) is created over the guide wire (not shown) with the first implant (10) now positioned to be driven into the bone fragments. FIG. 36B shows the first implant (10) in position within the distal bone fragment (4A). It can be appreciated that fragments (4a and 4b) based on the axis of the guide wire, are not properly configured for an ideal reduction of the fracture, as the bone fragments will make physical contact on one edge prior to the other bone surfaces properly mating.

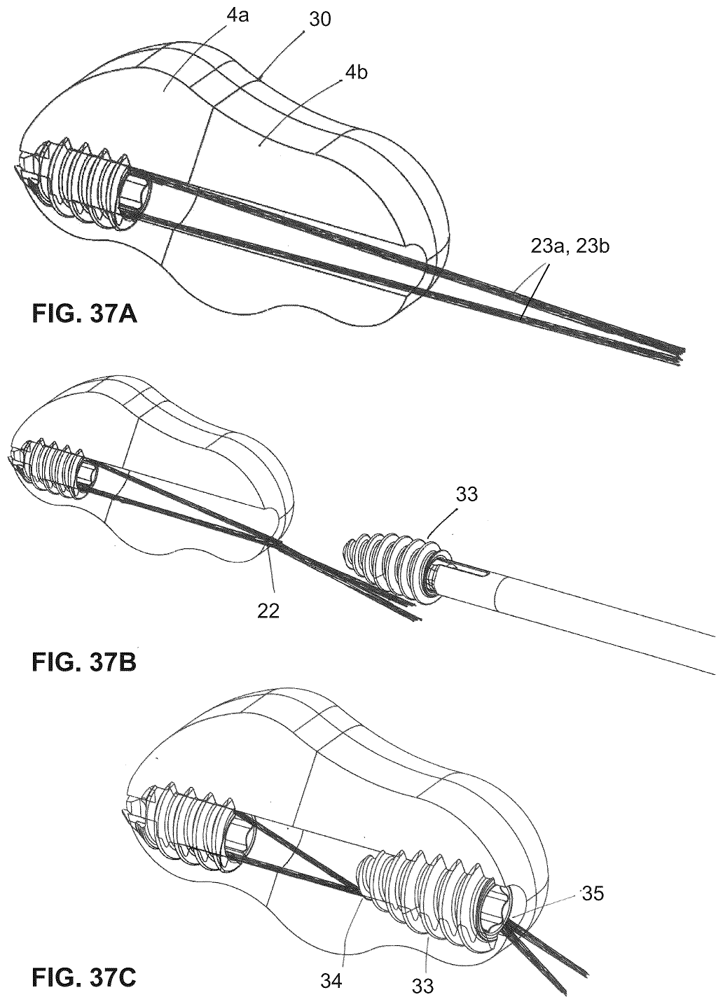

FIG. 36C illustrates the first implant (10) in position, and the driver (12) is axially retracted rearward leaving only the implant (10) in position in the distal fragment (4a) with only suture limbs (23a, 23b) trailing out of the pilot hole created in the proximal bone fragment (4b). Referring now to FIG. 37A, without wishing to be bound to a particular theory or mechanism, by applying a tensile load on suture limbs (23a, 23b), one fragment (4a) is allowed to translate, pivot, and/or align and settle into a more correctly reduced position relative to the remaining fragment (4b). Additionally, the bone fragments can approximate on a finer scale than if rigidly constrained, and improve proximity of existing bone and vascular networks that existed pre-fracture. The gap (5, 6) that was observed previously can now be closed more effectively.

In some embodiments, after applying a tensile load to the suture limbs (23a, 23b), a second implant (33) can be used to secure these suture limbs (23a, 23b) into place by way of radial compression. The second implant (33) is driven into the pilot hole, essentially locking down the suture limbs (23a, 23b) while they are being tensioned and can be driven flushed or below the surface of the bony segment. FIG. 37C shows the fully assembled system with the fracture reduced.

In some embodiments, an optional supporting implant (36) can be positioned in place within the aligned fragments prior to delivery of the second implant (33) to more fully fill in the pilot hole in the bone fragments. The suture would pass through this implant. Preferably, the supporting implant (10) is sized to a relatively smaller diameter than the pilot hole so as to avoid interfering with the expected misalignment of the guide wire holes and dislocating the fragments from the reduced position. This supporting implant (36) can be treated with traditional surface coatings consisting of spray applied porous titanium or ceramic based calcium sulphate or phosphate materials or a combination of these. This supporting implant (36) can also be treated with more recent surface treatments and nanotechnology treatments (BIOBraille™, Nanovis®), which can stimulate, signal, or provide an enhanced surface to adhere to the implant or to stimulate bone growth in the adjacent or surrounding areas.

Referring to FIG. 39, the lumen (38) of the supporting implant can be treated with one or more anti-clotting, antithrombogenic coatings (Parylene, PTFE, Liquiglide®, Vapor Deposition, Gore, etc.) to create an inner diameter of the implant so that it does not become occluded with blood clot. It is believed that in some bones, including the scaphoid, maintaining a vascular passageway may facilitate healing and continued vascularity of the bony segments.

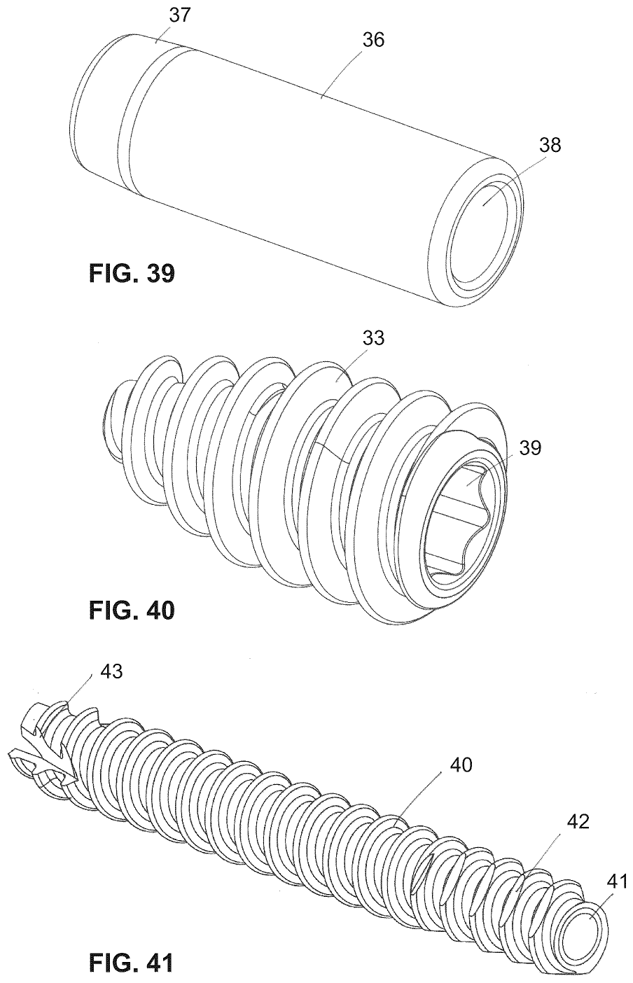

FIG. 40 is an illustration of the second implant (33) which creates a radial compression on the suture limbs (23a, 23b), or in other ways creates the tensile load in connection with the first implant (10). This second implant (33) may be of the same thread form and pitch as that of the first implant so as to minimize smearing and compromise of the threads that have been previously established in the bone segment as a result of passing the first implant (10) into position.

FIG. 41 illustrates an auxiliary implant (40) which may be used in conjunction with the reduction system for additional threaded fixation across the fracture plane. Without wishing to limit the present invention, as the reduction and compression has been achieved with the first and second implant sequence, no additional compression feature or functionality may be required on these auxiliary implants (40). As such, in some embodiments, they can be simple threaded rods or pins with a cutting and tapping distal tip. In this embodiment, an external tri-lobe drive (48) may be utilized so that they can be driven into the bone with a small diameter driver (44) that possesses a cutting tip (45) and can countersink these auxiliary implants (40) into the surface of the bone. These perimeter auxiliary implants (40) may also be treated with nano technologies or anti-thrombogenic inner diameter coatings.

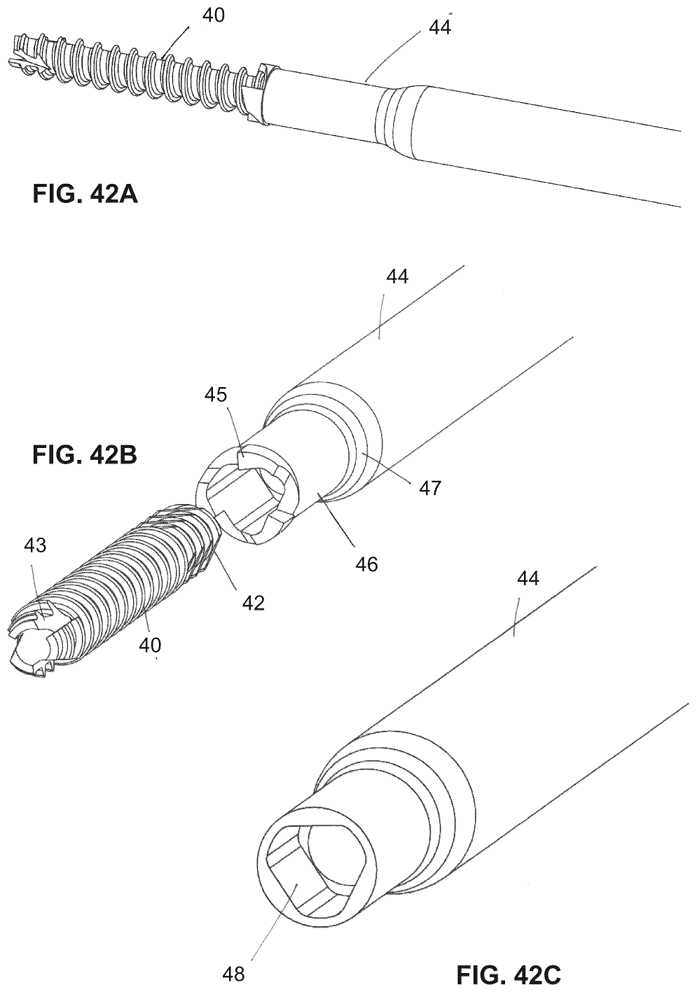

In one embodiment, FIG. 42A shows the external drive (44) for the auxiliary implants (40). In this configuration, multiple length sizes of the auxiliary implants (40) are not needed. It is only important that this threaded component be placed so that it is spanning the fracture plane and it is not intended to provide additional compression to the fracture site. FIG. 42B illustrates the distal tip of the driver which has sharp cutting tips (45) and a reduced diameter area (46) so that driver (44) can deliver the auxiliary implant (40) directly into the bone and recessed into the surface. FIG. 42C illustrates a tri-lobe driver configuration/interface (48) for use with the auxiliary implant (40).

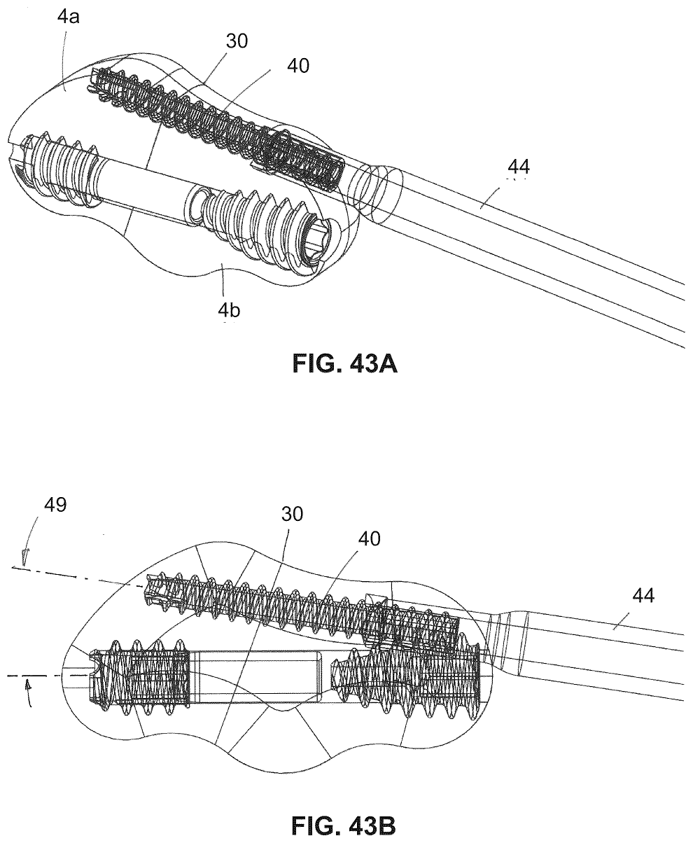

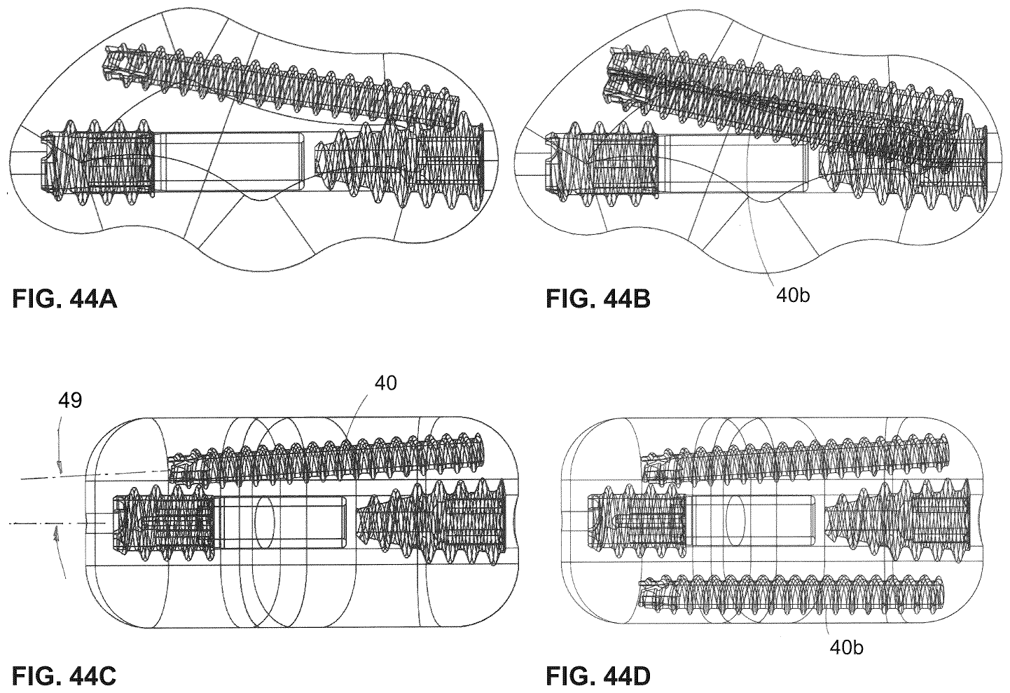

In some embodiments, as shown in FIG. 43A, reduction of the fracture site can be successfully accomplished with the first and second implants (10, 33). A supporting implant (36) has been placed and the auxiliary implant (40) has been driven into a separate remote segment of the bone across the fracture plane to further stabilize and provide rigid fixation across the fracture. By eliminating the need for auxiliary implant (40) to also try to create compression, the auxiliary implant (40) can be made much smaller than devices typically used for these fractures. FIGS. 43B and 44A-44D show additional embodiments of using the auxiliary implants (40). Without wishing to be bound to a particular theory or mechanism, by utilizing one or more auxiliary implants (40) at a variety of divergent angles and directions, a much more stable, axially and torsionally, fixation can be accomplished.

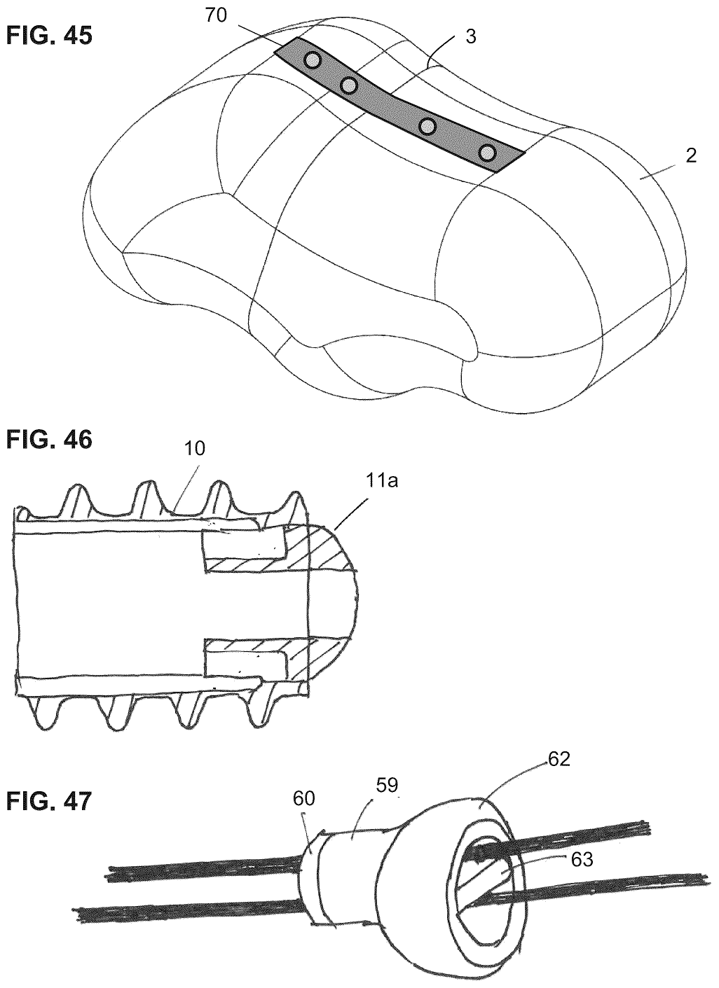

In alternative embodiments, as shown in FIG. 46, implants that are abbreviated, blunted or truncated distally may be utilized in thinner or smaller bone fragments or those that may be close to the surface of the skin or other structures.

In alternative embodiments, as shown in FIG. 47, a locking element (63) at the proximal end of the second implant (63) would allow for the tying of sliding and locking slipknots around said element. This embodiment would allow the first and second implants to be tensioned and pulled together without utilizing a threaded implant to lock the suture against the lumen of the bone.

In another alternative embodiment, not illustrated, the sutures may be disposed through a lumen of the second implant, and a plug or cap is inserted into said lumen, which presses the suture against a surface of the second implant and secures the suture in place.

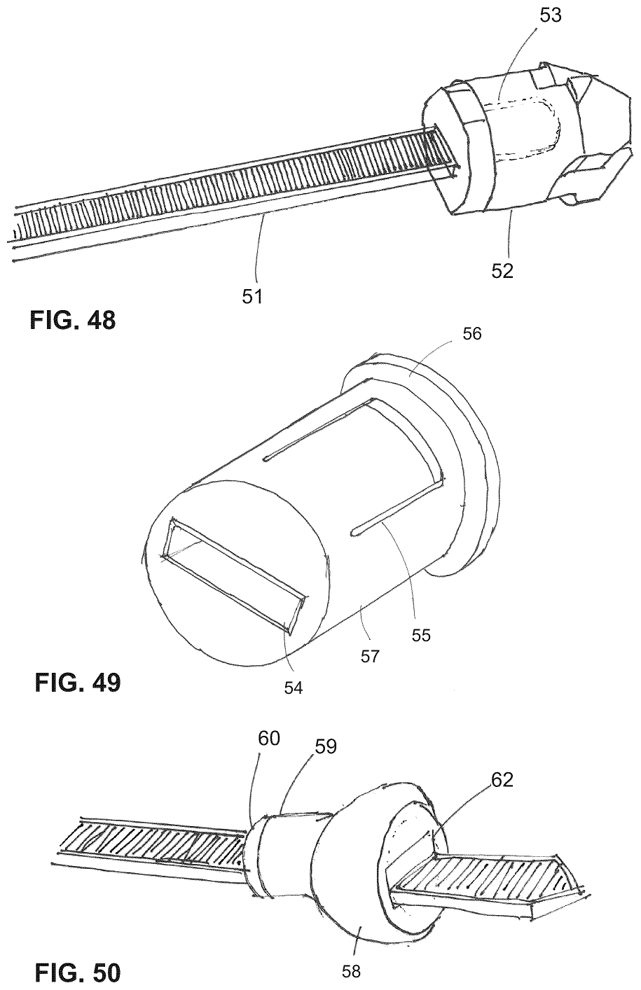

Alternative embodiments of performing surgical repair of a fracture are shown in FIGS. 48-50. In FIG. 48, the first implant (52) may have some other configuration, such as a zip-tie configuration (51) that allows for reduction and fixation between the first and second implants. The tail end of the zip-tie is locked into place by a zip-tie lock (55) in the second implant. In some embodiments, one or more zip-ties may be used between the first and second implants. For example, the first implant may include two zip-ties, and the second implant may include two zip-tie locks.

FIG. 49 shows a second implant with a flange element (56) which provides a tensile reaction force against the cortical surface of the bone or an additional implant element such as a bone washer or bone plate. FIG. 50 shows an alternative configuration that may include a boss (59). In some embodiments, as shown in FIG. 45, the boss (59) may interface with conventional plating systems or other traditional fixation devices so that this device can be used in fracture reduction in combination with fracture plates (70).

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

Reference numbers recited herein, in the drawings, and in the claims are solely for ease of examination of this patent application and are exemplary. The reference numbers are not intended in any way to limit the scope of the claims to the particular features having the corresponding reference numbers in the drawings.

What is claimed is:

1. A tissue or fracture fixation system (100), comprising:
a) a flexible tension component (150);
b) a first implant (110) comprising an attachment component (118) configured to couple the tension component (150) with the first implant (110); and
c) a second implant (130) configured to engage the tension component (150) to secure the first implant (110) relative to the second implant (130).

2. The system (100) of claim 1, wherein the first implant (110) further comprises a lumen (116) configured for use with a guidewire, wherein the attachment component (118) is offset from a central axis of the lumen, wherein the tension component (150) is coupled and routed through the first implant such that the tension component (150) does not interfere with the lumen (116) when used with the guidewire.

3. The system (100) of claim 1, wherein the first implant (110) is radially symmetric.

4. The system (100) of claim 1, wherein the attachment component (118) is a U-shaped groove, wherein the attachment component (118) acts as a pulley mechanism that engages the tension component (150).

5. The system (100) of claim 1, wherein the tension component (150) comprises a compressible locking component (155) configured to be disposed within the second implant (130), wherein the compressible locking component (155) comprises a sliding knot bundle.

6. The system (100) of claim 1, wherein the second implant (130) comprises a lumen (136) through which the tension component (150) is disposed, wherein the second implant (130) has an opening coupled to the lumen, wherein the opening is a tapering wedge from the proximal end into an interior of the lumen, wherein the opening of the second implant (130) is sized to receive and capture the compressible locking component (155).

7. The system (100) of claim 1, wherein the second implant (130) comprises a V-shaped notch (140) disposed on its distal end (132), wherein the V-shaped notch (140) is configured to capture a tissue, a suture, or suture tape.

8. The system (100) of claim 1, wherein the second implant (130) has a tapering outer diameter such that the second implant has a reduction in size at its distal end (132), wherein the second implant is configured to capture and drive a tissue, a suture, or suture tape into a hole, wherein the tapered distal end (132) allows for easier insertion of said tissue, suture, or suture tape by into said hole.

9. The system (100) of claim 1, wherein the second implant (130) has a threaded surface, barbs, or annular protrusions on its outer surface, and/or a ferrule (138) disposed at its proximal end (134).

10. The system (100) of claim 1, wherein the second implant (130) is T-shaped, wherein the second implant (130) comprises T-arms (138) disposed at its proximal end (134), wherein the T-shaped arms (138) are configured to anchor tissue (350).

11. The system (100) of claim 1, wherein the second implant (130) is L-shaped, wherein the second implant (130) has an arm (138) disposed at its proximal end (134) for anchoring tissue (350).

12. The system (100) of claim 1, further comprising at least one supporting implant (300) disposed between the first implant (110) and the second implant (130), wherein the tension component (150) is disposed through a lumen (310) of the supporting implant or adjacent to the supporting implant (300).

13. The system (100) of claim 12, wherein the first implant (110), the second implant (130), at least one supporting implant (300), or a combination thereof is treated with one or more anti-clotting, antithrombogenic coatings surface treatments, or nanotechnology treatments to stimulate bone growth.

14. The system (100) of claim 1, further comprising a driver tool (200) for implanting the first implant (110) and the second implant (130), wherein the driver tool (200) comprises a drive shaft (203) having a channel or lumen for receiving the tension component (150) and a drive distal tip, wherein the driver tool (200) comprises a handle (205) connected to the drive shaft (203).

15. The system (100) of claim 14, wherein the handle (205) comprises a receiving area (255) disposed within the handle and intersects a pathway for the tension component; a storage compartment (217); and grooves (215) that lead to said storage compartment (217); wherein the storage compartment (217) is configured to retain at least the second implant (130) prior to use, wherein the grooves (215) are configured to retain at least portion of the tension component (150) prior to use.

16. The system (100) of claim 15, further comprising a winder tool (250) configured to be inserted into the receiving area (255) of the handle, and capture and wind the tension component (150), wherein as the tension component (150) is wound onto the winder tool (250), tension is applied to the tension component (150).

17. The system of claim 14, wherein the driver tool (200) comprises a ferrule (260) connected to the drive shaft (203), wherein the ferrule (260) is configured to retain the second implant (130) prior to use.

18. A method of anchoring a first tissue portion (4a) to a second tissue portion (4b), comprising:
a) implanting a first implant (110) that is coupled to a tension component (150), via an attachment component (118), into the first tissue portion (4a); and
b) implanting at least a portion of a second implant (130) into the second tissue portion (4b), wherein the second implant (130) engages and secures the tension component (150) in place, wherein the second implant (130) captures a third tissue portion and fixates it to the second tissue portion (4b).

19. The method of claim 18, wherein the first tissue portion (4a) and the second tissue portion (4b) are bone, wherein the third tissue portion is a ligament or tendon.

20. The method of claim 18, wherein implanting comprises applying tension to the tension component (150) such that the second implant (130) engages a compressible locking component (155) of the tension component (150), which causes the compressible locking component (155) to collapse upon itself and secure the second implant (130) relative to the first implant (110).
